SEQUENCE LISTING (57 sequences)

SEQ ID NO: 1 - DNA, 678 bp, Artificial Sequence
  Synthetic DNA
atggctggttctcgcagaaagaaacatatccatgaaatcccgccccgaattcatatgtctagattagataaaagtaaagtgattaacagcgcattagagctgcttaatgaggtcggaatcgaaggtttaacaacccgtaaactcgcccagaagctaggtgtagagcagcctacattgtattggcatgtaaaaaataagcgggctttgctcgacgccttagccattgagatgttagataggcaccatactcacttttgccctttagaaggggaaagctggcaagattttttacgtaataaggctaaaagttttagatgtgctttactaagtcatcgcgatggagcaaaagtacatttaggtacacggcctacagaaaaacagtatgaaactctcgaaaatcaattagcctttttatgccaacaaggtttttcactagagaatgcattatatgcactcagcgctgtggggcattttactttaggttgcgtattggaagatcaagagcatcaagtcgctaaagaagaaagggaaacacctactactgatagtatgccgccattattacgacaagctatcgaattatttgatcaccaaggtgcagagccagccttcttattcggccttgaattgatcatatgcggattagaaaaacaacttaaatgtgaaagtgggtcttaa

SEQ ID NO: 2 - DNA, 31 bp, Artificial Sequence
  Synthetic DNA
gagctctctagaactagtggatcctgcagga

SEQ ID NO: 3 - DNA, 48 bp, Artificial Sequence
  Synthetic DNA
ctagcataaccccttggggcctctaaacgggtcttgaggggttttttg

SEQ ID NO: 4 - DNA, 25 bp, Artificial Sequence
  Synthetic DNA
ggaattcgatatcactcgaggtacc

SEQ ID NO: 5 - DNA, 87 bp, Artificial Sequence
  Synthetic DNA
caaataaaacgaaaggctcagtcgaaagactgggcctttcgttttatctgttgtttgtcggtgaacgctctcctgagtaggacaaat

SEQ ID NO: 6 - DNA, 3414 bp, Artificial Sequence
  Synthetic DNA
atgaaaaaactgctggtcgcaaatcgtggagaaatcgccgttcgtgtctttcgtgcctgtaatgaactcggactttctacagtagccgtctatgcacgtgaagatgaatattccgttcatcgctttaaagcagatgaatcttaccttatcggtcaaggtaaaaaaccaattgatgcttatttggatattgatgatattattcgtgttgctcttgaatcaggagcagatgccattcatccgggttatggtcttttatctgaaaatcttgaatttgctacaaaagttcgtgcagcaggattagtttttgtcggtcctgaacttcatcatttggatattttcggcgataaaatcaaagcaaaagccgcagctgatgaagctcaagttccgggcattccgggaacaaatggtgcagtagatattgacggcgctcttgaatttgctcaaacttacggatatccagtcatgattaaggcagcattgggcggcggcggtcgtggaatgcgtgttgcgcgtaatgacgctgaaatgcacgacggatatgctcgtgcgaaatcagaagctatcggtgcctttggttctggagaaatctatgttgaaaaatacattgaaaatcctaagcatattgaagttcaaattcttggggatagtcatggaaatattgtccatttgcacgaacgtgattgctctgtccaacgccgtaatcaaaaagtcattgaaattgctccagccgtaggactctcaccagagttccgtaatgaaatttgtgaagcagcagttaaactttgtaaaaatgttggctatgttaatgctgggacggttgaatttttagtcaaagatgataagttctactttatcgaagtcaacccacgtgttcaagttgaacacacaattaccgagcttattacaggtgtagatattgttcaagcacaaattttgattgctcaaggcaaagatttacatacagaaattggtatcccggcacaagctgaaattccacttttgggctcagccattcaatgtcgtattactacagaagacccgcaaaatggcttcttgccagatacaggtaaaatcgatacctaccgttcaccaggtggtttcggcattcgtttggacgttggaaatgcctatgctggttatgaagtgactccgtattttgactctcttttagtaaaagtttgtacctttgctaatgaatttagcgatagtgtacgtaaaatggatcgtgtgcttcatgaatttcgtattcgtggggtgaaaactaatattccatttttgattaatgttattgccaatgaaaactttacgagcggacaagcaacaacaacctttattgacaatactccaagtcttttcaatttcccacgcttacgtgaccgtggaacaaaaaccttacactacttatcaatgattacagtcaatggtttcccagggattgaaaatacagaaaaacgccattttgaagaacctcgtcaacctctgcttaacattgaaaagaaaaagacagctaaaaatatcttagatgaacaaggggctgatgcggtagttgaatatgtgaaaaatacaaaagaagtattattgacagatacaactttacgtgatgctcaccagtctcttcttgccactcgtttgcgtttgcaagatatgaaaggaattgctcaagccattgaccaaggacttccagaacttttctcagctgaaatgtggggtggggcaacctttgatgtcgcttatcgtttcttgaatgaatcgccttggtatcgtctgcgtaaattacgtaaactcatgccaaataccatgttccaaatgcttttccgtggttcaaatgcagttggatatcaaaactatcctgataatgtcattgaagaatttatccacgtagctgcacatgaaggaatcgatgtctttcgtatctttgatagcctcaactggttgccacaaatggaaaaatcaatccaagcagtgcgtgataatggaaaaattgccgaagcaaccatttgttatacaggagatatccttgacccaagtcgtccaaaatataatatccaatactacaaagatttggcaaaagagttagaagctactggggctcatatccttgccgttaaagatatggcgggcttgttgaaacctcaagcggcatatcgcttgatttcagaattaaaagatacggttgacttaccaattcacttgcatacacatgatacttcaggaaatggtattattacctattctggtgcaactcaagcaggagtagatattattgatgtggcaactgccagtcttgctggtggaacttctcaaccttcaatgcaatcaatttattatgcccttgaacatggtccgcgtcatgcttcaattaatgtgaaaaatgcagagcaaattgaccattattgggaagatgtgcgtaaatattatgcaccttttgaggcaggaattacgagcccacaaactgaagtttacatgcatgaaatgcctggcggacaatatactaacttgaaatctcaagcagcagctgttggacttggacatcgttttgatgaaatcaaacaaatgtatcgtaaagtaaacatgatgtttggcgatatcattaaagtaactccttcatcaaaagtagttggtgatatggcactctttatgattcaaaacgaattgacagaagaggatgtctatgcgcgtggaaatgagcttaacttccctgaatcagtagtctcattcttccgtggtgatttaggacagcctgttggaggtttcccagaagaactgcaaaaaattattgtaaaagacaaatcggtcattatggatcgtccaggattacatgccgaaaaagttgattttgcaactgtaaaagctgacttggaacaaaaaattggttatgaaccaggtgatcatgaagttatctcttacattatgtatccacaagttttccttgattatcaaaaaatgcaacgtgaatttggagctgtcacactgctcgatactccaactttcttacacggaatgcgcctcaatgaaaaaattgaagtccaaattgaaaaaggtaaaacgctcagcattcgtttagatgaaattggagaacctgacctcgctggaaatcgtgtgctcttctttaacttgaacggtcagcgtcgtgaagttgttattaatgaccaatccgttcaaactcaaattgtagctaaacgtaaggccgaaacaggtaatccaaaccaaattggagcaactatgccgggttctgttcttgaaatcctggttaaagctggagataaagttaaaaaaggacaagctttgatggttactgaagccatgaagatggaaacgaccattgagtcaccatttgatggagaggttattgcccttcatgttgtcaaaggtgaagccattcaaacacaagacttattgattgaaattgactaa

SEQ ID NO: 7 - DNA, 20 bp, Artificial Sequence
  Synthetic DNA
ttaaagaggagaaaggtacc

SEQ ID NO: 8 - DNA, 20 bp, Artificial Sequence
  Synthetic DNA
aaacgaagcattgggatctt

SEQ ID NO: 9 - DNA, 19 bp, Artificial Sequence
  Synthetic DNA
tacacaggaaacctactag

SEQ ID NO: 10 - protein, 10 aa, Artificial Sequence
  Protein translated from synthetic DNA
AADENYALAA

SEQ ID NO: 11 - DNA, 33 bp, Artificial Sequence
  Synthetic DNA
gcagcaaacgacgaaaactacgctttagcagct

SEQ ID NO: 12 - DNA, 4249 bp, Artificial Sequence
  Synthetic DNA
gctagcagcacgccatagtgactggcgatgctgtcggaatggacgataattcgacagtaagacgggtaagcctgttgatgataccgctgccttactgggtgcattagccagtctgaatgacctgtcacgggataatccgaagtggtcagactggaaaatcagagggcaggaactgcgaacagcaaaaagtcagatagcaccacatagcagacccgccataaaacgccctgagagcccgtgacgggcttttcttgtattatgggtagtttccttgcatgaatccataaaaggcgcctgtagtgccatttacccccattcactgccagagccgtgagcgcagcgaactgaatgtcacgaaaaagacagcgactcaggtgcctgatggtcggagacaaaaggaatattcagcgatttgcccgagaattatcccgcaagaggcccggcagtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattgccggcgatagcttggctgcaggtgatgattatcagccagcagagattaaggaaaacagacaggtttattgagcgcttatctttccctttatttttgctgcggtaagtcgcataaaaaccattcttcataattcaatccatttactatgttatgttctgaggggagtgaaaattcccctaattcgatgaagattcttgctcaattgttatcagctatgcgccgaccagaacaccttgccgatcagccaaacgtctcttcaggccactgactagcgataactttccccacaacggaacaactctcattgcatgggatcattgggtactgtgggtttagtggttgtaaaaacacctgaccgctatccctgatcagtttcttgaaggtaaactcatcacccccaagtctggctatgcagaaatcacctggctcaacagcctgctcagggtcaacgagaattaacattccgtcaggaaagcttggcttggagcctgttggtgcggtcatggaattaccttcaacctcaagccagaatgcagaatcactggcttttttggttgtgcttacccatctctccgcatcacctttggtaaaggttctaagcttaggtgagaacatccctgcctgaacatgagaaaaaacagggtactcatactcacttctaagtgacggctgcatactaaccgcttcatacatctcgtagatttctctggcgattgaagggctaaattcttcaacgctaactttgagaatttttgcaagcaatgcggcgttataagcatttaatgcattgatgccattaaataaagcaccaacgcctgactgccccatccccatcttgtctgcgacagattcctgggataagccaagttcatttttctttttttcataaattgctttaaggcgacgtgcgtcctcaagctgctcttgtgttaatggtttcttttttgtgctcatacgttaaatctatcaccgcaagggataaatatctaacaccgtgcgtgttgactattttacctctggcggtgataatggttgcatgtactaaggaggttgtatggaacaacgcataaccctgaaagattatgcaatgcgctttgggcaaaccaagacagctaaagatctctcacctaccaaacaatgcccccctgcaaaaaataaattcatataaaaaacatacagataaccatctgcggtgataaattatctctggcggtgttgacataaataccactggcggtgatactgagcacatcagcaggacgcactgaccaccatgaaggtgacgctcttaaaaattaagccctgaagaagggcagcattcaaagcagaaggctttggggtgtgtgatacgaaacgaagcattgggatcctaaggaggttaactatgacgtcgacgcgtctgcagaagcttctagaattcgagctcccgggtaccatggcatgcatcgatagatctcgaggcctcgcgagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggc

SEQ ID NO: 13 - DNA, 55 bp, Artificial Sequence
  Synthetic DNA
tccctatcagtgatagagattgacatccctatcagtgatagagatactgagcaca

SEQ ID NO: 14 - DNA, 3075 bp, Artificial Sequence
  Synthetic DNA
atgaccatgattacggattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtgacctatcccattacggtcaatccgccgtttgttcccacggagaatccgacgggttgttactcgctcacatttaatgttgatgaaagctggctacaggaaggccagacgcgaattatttttgatggcgttaactcggcgtttcatctgtggtgcaacgggcgctgggtcggttacggccaggacagtcgtttgccgtctgaatttgacctgagcgcatttttacgcgccggagaaaaccgcctcgcggtgatggtgctgcgctggagtgacggcagttatctggaagatcaggatatgtggcggatgagcggcattttccgtgacgtctcgttgctgcataaaccgactacacaaatcagcgatttccatgttgccactcgctttaatgatgatttcagccgcgctgtactggaggctgaagttcagatgtgcggcgagttgcgtgactacctacgggtaacagtttctttatggcagggtgaaacgcaggtcgccagcggcaccgcgcctttcggcggtgaaattatcgatgagcgtggtggttatgccgatcgcgtcacactacgtctgaacgtcgaaaacccgaaactgtggagcgccgaaatcccgaatctctatcgtgcggtggttgaactgcacaccgccgacggcacgctgattgaagcagaagcctgcgatgtcggtttccgcgaggtgcggattgaaaatggtctgctgctgctgaacggcaagccgttgctgattcgaggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctaccggcgatgagcgaacgcgtaacgcgaatggtgcagcgcgatcgtaatcacccgagtgtgatcatctggtcgctggggaatgaatcaggccacggcgctaatcacgacgcgctgtatcgctggatcaaatctgtcgatccttcccgcccggtgcagtatgaaggcggcggagccgacaccacggccaccgatattatttgcccgatgtacgcgcgcgtggatgaagaccagcccttcccggctgtgccgaaatggtccatcaaaaaatggctttcgctacctggagagacgcgcccgctgatcctttgcgaatacgcccacgcgatgggtaacagtcttggcggtttcgctaaatactggcaggcgtttcgtcagtatccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccagcgctgacggaagcaaaacaccagcagcagtttttccagttccgtttatccgggcaaaccatcgaagtgaccagcgaatacctgttccgtcatagcgataacgagctcctgcactggatggtggcgctggatggtaagccgctggcaagcggtgaagtgcctctggatgtcgctccacaaggtaaacagttgattgaactgcctgaactaccgcagccggagagcgccgggcaactctggctcacagtacgcgtagtgcaaccgaacgcgaccgcatggtcagaagccgggcacatcagcgcctggcagcagtggcgtctggcggaaaacctcagtgtgacgctccccgccgcgtcccacgccatcccgcatctgaccaccagcgaaatggatttttgcatcgagctgggtaataagcgttggcaatttaaccgccagtcaggctttctttcacagatgtggattggcgataaaaaacaactgctgacgccgctgcgcgatcagttcacccgtgcaccgctggataacgacattggcgtaagtgaagcgacccgcattgaccctaacgcctgggtcgaacgctggaaggcggcgggccattaccaggccgaagcagcgttgttgcagtgcacggcagatacacttgctgatgcggtgctgattacgaccgctcacgcgtggcagcatcaggggaaaaccttatttatcagccggaaaacctaccggattgatggtagtggtcaaatggcgattaccgttgatgttgaagtggcgagcgatacaccgcatccggcgcggattggcctgaactgccagctggcgcaggtagcagagcgggtaaactggctcggattagggccgcaagaaaactatcccgaccgccttactgccgcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagtcaacagcaactgatggaaaccagccatcgccatctgctgcacgcggaagaaggcacatggctgaatatcgacggtttccatatggggattggtggcgacgactcctggagcccgtcagtatcggcggaattccagctgagcgccggtcgctaccattaccagttggtctggtgtcaaaaataa

SEQ ID NO: 15 - DNA, 1083 bp, Artificial Sequence
  Synthetic DNA
gtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtga

SEQ ID NO: 16 - DNA, 621 bp, Artificial Sequence
  Synthetic DNA
atgttgatgttatttctcaccgtcgccatggtgcacattgtggcgcttatgagccccggtcccgatttcttttttgtctctcagaccgctgtcagtcgttcccgtaaagaagcgatgatgggcgtgctgggcattacctgcggcgtaatggtttgggctgggattgcgctgcttggcctgcatttgattatcgaaaaaatggcctggctgcatacgctgattatggtgggcggtggcctgtatctctgctggatgggttaccagatgctacgtggtgcactgaaaaaagaggcggtttctgcacctgcgccacaggtcgagctggcgaaaagtgggcgcagtttcctgaaaggtttactgaccaatctcgctaatccgaaagcgattatctactttggctcggtgttctcattgtttgtcggtgataacgttggcactaccgcgcgctggggcatttttgcgctgatcattgtcgaaacgctggcgtggtttaccgtcgttgccagcctgtttgccctgccgcaaatgcgccgtggttatcaacgtctggcgaagtggattgatggttttgccggggcgttatttgccggatttggcattcatttgattatttcgcggtga

SEQ ID NO: 17 - DNA, 3414 bp, Artificial Sequence
  Synthetic DNA
atgaaaaaactactcgtcgccaatcgtggagaaatcgccgttcgtgtctttcgtgcctgtaatgaactcggactttctacagtagccgtctatgcaagagaagatgaatattccgttcatcgctttaaagcagatgaatcttaccttatcggtcaaggtaaaaaaccaattgatgcttatttggatattgatgatattattcgtgttgctcttgaatcaggagcagatgccattcatcccggttatggtcttttatctgaaaatcttgaatttgctacaaaagttcgagcagcaggattagtttttgtcggtcctgaacttcatcatttggatattttcggcgataaaatcaaagcaaaagccgcagctgatgaagctcaagttcccggaattcccggaacaaatggtgcagtagatattgacggagctcttgaatttgctcaaacttacggatatccagtcatgattaaggcagcattgggcggcggcggtcgtggaatgcgtgttgcgcgtaatgacgctgaaatgcacgacggatatgctcgtgcgaaatcagaagctatcggtgcctttggttctggagaaatctatgttgaaaaatacattgaaaatcctaagcatattgaagttcaaattcttggggatagtcatggaaatattgtccatttgcacgaacgtgattgctctgtccaacgccgaaatcaaaaagtcattgaaattgctccagccgtaggactctcaccagagttccgtaatgaaatttgtgaagcagcagttaaactttgtaaaaatgttggctatgttaatgctgggacggttgaatttttagtcaaagatgataagttctactttatcgaagtcaacccacgtgttcaagttgaacacacaattaccgagcttattacaggtgtagatattgttcaagcacaaattttgattgctcaaggcaaagatttacatacagaaattggtatcccggcacaagctgaaataccacttttgggctcagccattcaatgtcgtattactacagaagacccgcaaaatggcttcttgccagatacaggtaaaatcgatacctaccgttcaccaggtggtttcggcattcgtttggacgttggaaatgcctatgctggttatgaagtgactccctattttgactcgcttttagtaaaagtttgtacctttgctaatgaatttagcgatagtgtacgtaaaatggatcgtgtgcttcatgaattccgtattcgtggggtgaaaactaatattccatttttgattaatgttattgccaatgaaaactttacgagcggacaagcaacaacaacctttattgacaatactccaagtcttttcaatttcccacgcttacgtgaccgtggaacaaaaaccttacactacttatcaatgattacagtcaatggtttcccagggattgaaaatacagaaaaacgccattttgaagaacctcgtcaacctctacttaacattgaaaagaaaaagacagctaaaaatatcttagatgaacaaggggctgatgcggtagttgaatatgtgaaaaatacaaaagaagtattattgacagatacaactttacgtgatgctcaccagtctcttcttgccactcgtttgcgtttgcaagatatgaaaggaattgctcaagccattgaccaaggacttccagaacttttctcagctgaaatgtggggtggggcaacctttgatgtcgcttatcgtttcttgaatgaatcgccttggtatcgtctacgtaaattacgtaaactcatgccaaataccatgttccaaatgcttttccgtggttcaaatgcagttggatatcaaaactatcctgataatgtcattgaagaatttatccacgtagctgcacatgaaggaatcgatgtctttcgtatctttgatagcctcaactggttgccacaaatggaaaaatcaatccaagcagtgcgtgataatggaaaaattgccgaagcaaccatttgttatacaggagatatccttgacccaagtcgaccaaaatataatatccaatactacaaagatttggcaaaagagttagaagctactggggctcatatacttgccgttaaagatatggcgggcttgttgaaacctcaagcggcatatcgcttgatttcagaattaaaagatacggttgacttaccaattcacttgcatacacatgatacttcaggaaatggtattattacctattctggtgcaactcaagcaggagtagatattattgatgtggcaactgccagtcttgctggtggaacttctcaaccttcaatgcaatcaatttattatgcccttgaacatggtccccgtcatgcttcaattaatgtgaaaaatgcagagcaaattgaccattattgggaagatgtgcgtaaatattatgcaccttttgaggcaggaattacgagcccacaaactgaagtttacatgcatgaaatgcctggcggacaatatactaacttgaaatctcaagcagcagctgttggacttggacatcgttttgatgaaatcaaacaaatgtatcgtaaagtaaacatgatgtttggcgatatcattaaagtaactccttcatcaaaagtagttggtgatatggcactctttatgattcaaaacgaattgacagaagaggatgtctatgcgcgaggaaatgagcttaacttccctgaatcagtagtctcattcttccgtggtgatttaggacagcctgttggaggtttcccagaagaactacaaaaaattattgtaaaagacaaatcggtcattatggatcgtccaggattacatgccgaaaaagttgattttgcaactgtaaaagctgacttggaacaaaaaattggttatgaaccaggtgatcatgaagttatctcttacattatgtatccacaagttttccttgattatcaaaaaatgcaaagagaatttggagctgtcacactactcgatactccaactttcttacacggaatgcgcctcaatgaaaaaattgaagtccaaattgaaaaaggtaaaacgctcagcattcgtttagatgaaataggagaacctgacctcgctggaaatcgtgtgctcttctttaacttgaacggtcagcgtcgtgaagttgttattaatgaccaatccgttcaaactcaaattgtagctaaacgtaaggccgaaacaggtaatccaaaccaaattggagcaactatgcccggttctgttcttgaaatcctagttaaagctggagataaagttaaaaaaggacaagctttgatggttactgaagccatgaagatggaaacgaccattgagtcaccatttgatggagaggttattgcccttcatgttgtcaaaggtgaagccattcaaacacaagacttattgattgaaattgactaa

SEQ ID NO: 18 - DNA, 1533 bp, Artificial Sequence
  Synthetic DNA
atgcgaattggcataccaagagaacggttaaccaatgaaacccgtgttgcagcaacgccaaaaacagtggaacagctgctgaaactgggttttaccgtcgcggtagagagcggcgcgggtcaactggcaagttttgacgataaagcgtttgtgcaagcgggcgctgaaattgtagaagggaatagcgtctggcagtcagagatcattctgaaggtcaatgcgccgttagatgatgaaattgcgttactgaatcctgggacaacgctggtgagttttatctggcctgcgcagaatccggaattaatgcaaaaacttgcggaacgtaacgtgaccgtgatggcgatggactctgtgccgcgtatctcacgcgcacaatcgctggacgcactaagctcgatggcgaacatcgccggttatcgcgccattgttgaagcggcacatgaatttgggcgcttctttaccgggcaaattactgcggccgggaaagtgccaccggcaaaagtgatggtgattggtgcgggtgttgcaggtctggccgccattggcgcagcaaacagtctcggcgcgattgtgcgtgcattcgacacccgcccggaagtgaaagaacaagttcaaagtatgggcgcggaattcctcgagctggattttaaagaggaagctggcagcggcgatggctatgccaaagtgatgtcggacgcgttcatcaaagcggaaatggaactctttgccgcccaggcaaaagaggtcgatatcattgtcaccaccgcgcttattccaggcaaaccagcgccgaagctaattacccgtgaaatggttgactccatgaaggcgggcagtgtgattgtcgacctggcagcccaaaacggcggcaactgtgaatacaccgtgccgggtgaaatcttcactacggaaaatggtgtcaaagtgattggttataccgatcttccgggccgtctgccgacgcaatcctcacagctttacggcacaaacctcgttaatctgctgaaactgttgtgcaaagagaaagacggcaatatcactgttgattttgatgatgtggtgattcgcggcgtgaccgtgatccgtgcgggcgaaattacctggccggcaccgccgattcaggtatcagctcagccgcaggcggcacaaaaagcggcaccggaagtgaaaactgaggaaaaatgtacctgctcaccgtggcgtaaatacgcgttgatggcgctggcaatcattctttttggctggatggcaagcgttgcgccgaaagaattccttgggcacttcaccgttttcgcgctggcctgcgttgtcggttattacgtggtgtggaatgtatcgcacgcgctgcatacaccgttgatgtcggtcaccaacgcgatttcagggattattgttgtcggagcactgttgcagattggccagggcggctgggttagcttccttagttttatcgcggtgcttatagccagcattaatattttcggtggcttcaccgtgactcagcgcatgctgaaaatgttccgcaaaaattaa

SEQ ID NO: 19 - DNA, 1389 bp, Artificial Sequence
  Synthetic DNA
atgtctggaggattagttacagctgcatacattgttgccgcgatcctgtttatcttcagtctggccggtctttcgaaacatgaaacgtctcgccagggtaacaacttcggtatcgccgggatggcgattgcgttaatcgcaaccatttttggaccggatacgggtaatgttggctggatcttgctggcgatggtcattggtggggcaattggtatccgtctggcgaagaaagttgaaatgaccgaaatgccagaactggtggcgatcctgcatagcttcgtgggtctggcggcagtgctggttggctttaacagctatctgcatcatgacgcgggaatggcaccgattctggtcaatattcacctgacggaagtgttcctcggtatcttcatcggggcggtaacgttcacgggttcggtggtggcgttcggcaaactgtgtggcaagatttcgtctaaaccattgatgctgccaaaccgtcacaaaatgaacctggcggctctggtcgtttccttcctgctgctgattgtatttgttcgcacggacagcgtcggcctgcaagtgctggcattgctgataatgaccgcaattgcgctggtattcggctggcatttagtcgcctccatcggtggtgcagatatgccagtggtggtgtcgatgctgaactcgtactccggctgggcggctgcggctgcgggctttatgctcagcaacgacctgctgattgtgaccggtgcgctggtcggttcttcgggggctatcctttcttacattatgtgtaaggcgatgaaccgttcctttatcagcgttattgcgggtggtttcggcaccgacggctcttctactggcgatgatcaggaagtgggtgagcaccgcgaaatcaccgcagaagagacagcggaactgctgaaaaactcccattcagtgatcattactccggggtacggcatggcagtcgcgcaggcgcaatatcctgtcgctgaaattactgagaaattgcgcgctcgtggtattaatgtgcgtttcggtatccacccggtcgcggggcgtttgcctggacatatgaacgtattgctggctgaagcaaaagtaccgtatgacatcgtgctggaaatggacgagatcaatgatgactttgctgataccgataccgtactggtgattggtgctaacgatacggttaacccggcggcgcaggatgatccgaagagtccgattgctggtatgcctgtgctggaagtgtggaaagcgcagaacgtgattgtctttaaacgttcgatgaacactggctatgctggtgtgcaaaacccgctgttcttcaaggaaaacacccacatgctgtttggtgacgccaaagccagcgtggatgcaatcctgaaagctctgtaa

SEQ ID NO: 20 - DNA, 2652 bp, Artificial Sequence
  Synthetic DNA
atgaacgaacaatattccgcattgcgtagtaatgtcagtatgctcggcaaagtgctgggagaaaccatcaaggatgcgttgggagaacacattcttgaacgcgtagaaactatccgtaagttgtcgaaatcttcacgcgctggcaatgatgctaaccgccaggagttgctcaccaccttacaaaatttgtcgaacgacgagctgctgcccgttgcgcgtgcgtttagtcagttcctgaacctggccaacaccgccgagcaataccacagcatttcgccgaaaggcgaagctgccagcaacccggaagtgatcgcccgcaccctgcgtaaactgaaaaaccagccggaactgagcgaagacaccatcaaaaaagcagtggaatcgctgtcgctggaactggtcctcacggctcacccaaccgaaattacccgtcgtacactgatccacaaaatggtggaagtgaacgcctgtttaaaacagctcgataacaaagatatcgctgactacgaacacaaccagctgatgcgtcgcctgcgccagttgatcgcccagtcatggcataccgatgaaatccgtaagctgcgtccaagcccggtagatgaagccaaatggggctttgccgtagtggaaaacagcctgtggcaaggcgtaccaaattacctgcgcgaactgaacgaacaactggaagagaacctcggctacaaactgcccgtcgaatttgttccggtccgttttacttcgtggatgggcggcgaccgcgacggcaacccgaacgtcactgccgatatcacccgccacgtcctgctactcagccgctggaaagccaccgatttgttcctgaaagatattcaggtgctggtttctgaactgtcgatggttgaagcgacccctgaactgctggcgctggttggcgaagaaggtgccgcagaaccgtatcgctatctgatgaaaaacctgcgttctcgcctgatggcgacacaggcatggctggaagcgcgcctgaaaggcgaagaactgccaaaaccagaaggcctgctgacacaaaacgaagaactgtgggaaccgctctacgcttgctaccagtcacttcaggcgtgtggcatgggtattatcgccaacggcgatctgctcgacaccctgcgccgcgtgaaatgtttcggcgtaccgctggtccgtattgatatccgtcaggagagcacgcgtcataccgaagcgctgggcgagctgacccgctacctcggtatcggcgactacgaaagctggtcagaggccgacaaacaggcgttcctgatccgcgaactgaactccaaacgtccgcttctgccgcgcaactggcaaccaagcgccgaaacgcgcgaagtgctcgatacctgccaggtgattgccgaagcaccgcaaggctccattgccgcctacgtgatctcgatggcgaaaacgccgtccgacgtactggctgtccacctgctgctgaaagaagcgggtatcgggtttgcgatgccggttgctccgctgtttgaaaccctcgatgatctgaacaacgccaacgatgtcatgacccagctgctcaatattgactggtatcgtggcctgattcagggcaaacagatggtgatgattggctattccgactcagcaaaagatgcgggagtgatggcagcttcctgggcgcaatatcaggcacaggatgcattaatcaaaacctgcgaaaaagcgggtattgagctgacgttgttccacggtcgcggcggttccattggtcgcggcggcgcacctgctcatgcggcgctgctgtcacaaccgccaggaagcctgaaaggcggcctgcgcgtaaccgaacagggcgagatgatccgctttaaatatggtctgccagaaatcaccgtcagcagcctgtcgctttataccggggcgattctggaagccaacctgctgccaccgccggagccgaaagagagctggcgtcgcattatggatgaactgtcagtcatctcctgcgatgtctaccgcggctacgtacgtgaaaacaaagattttgtgccttacttccgctccgctacgccggaacaagaactgggcaaactgccgttgggttcacgtccggcgaaacgtcgcccaaccggcggcgtcgagtcactacgcgccattccgtggatcttcgcctggacgcaaaaccgtctgatgctccccgcctggctgggtgcaggtacggcgctgcaaaaagtggtcgaagacggcaaacagagcgagctggaggctatgtgccgcgattggccattcttctcgacgcgtctcggcatgctggagatggtcttcgccaaagcagacctgtggctggcggaatactatgaccaacgcctggtagacaaagcactgtggccgttaggtaaagagttacgcaacctgcaagaagaagacatcaaagtggtgctggcgattgccaacgattcccatctgatggccgatctgccgtggattgcagagtctattcagctacggaatatttacaccgacccgctgaacgtattgcaggccgagttgctgcaccgctcccgccaggcagaaaaagaaggccaggaaccggatcctcgcgtcgaacaagcgttaatggtcactattgccgggattgcggcaggtatgcgtaataccggctaa

SEQ ID NO: 21 - DNA, 1191 bp, Artificial Sequence
  Synthetic DNA
atgtttgagaacattaccgccgctcctgccgacccgattctgggcctggccgatctgtttcgtgccgatgaacgtcccggcaaaattaacctcgggattggtgtctataaagatgagacgggcaaaaccccggtactgaccagcgtgaaaaaggctgaacagtatctgctcgaaaatgaaaccaccaaaaattacctcggcattgacggcatccctgaatttggtcgctgcactcaggaactgctgtttggtaaaggtagcgccctgatcaatgacaaacgtgctcgcacggcacagactccggggggcactggcgcactacgcgtggctgccgatttcctggcaaaaaataccagcgttaagcgtgtgtgggtgagcaacccaagctggccgaaccataagagcgtctttaactctgcaggtctggaagttcgtgaatacgcttattatgatgcggaaaatcacactcttgacttcgatgcactgattaacagcctgaatgaagctcaggctggcgacgtagtgctgttccatggctgctgccataacccaaccggtatcgaccctacgctggaacaatggcaaacactggcacaactctccgttgagaaaggctggttaccgctgtttgacttcgcttaccagggttttgcccgtggtctggaagaagatgctgaaggactgcgcgctttcgcggctatgcataaagagctgattgttgccagttcctactctaaaaactttggcctgtacaacgagcgtgttggcgcttgtactctggttgctgccgacagtgaaaccgttgatcgcgcattcagccaaatgaaagcggcgattcgcgctaactactctaacccaccagcacacggcgcttctgttgttgccaccatcctgagcaacgatgcgttacgtgcgatttgggaacaagagctgactgatatgcgccagcgtattcagcgtatgcgtcagttgttcgtcaatacgctgcaggaaaaaggcgcaaaccgcgacttcagctttatcatcaaacagaacggcatgttctccttcagtggcctgacaaaagaacaagtgctgcgtctgcgcgaagagtttggcgtatatgcggttgcttctggtcgcgtaaatgtggccgggatgacaccagataacatggctccgctgtgcgaagcgattgtggcagtgctgtaa

SEQ ID NO: 22 - DNA, 1350 bp, Artificial Sequence
  Synthetic DNA
atgtctgaaattgttgtctccaaatttggcggtaccagcgtagctgattttgacgccatgaaccgcagcgctgatattgtgctttctgatgccaacgtgcgtttagttgtcctctcggcttctgctggtatcactaatctgctggtcgctttagctgaaggactggaacctggcgagcgattcgaaaaactcgacgctatccgcaacatccagtttgccattctggaacgtctgcgttacccgaacgttatccgtgaagagattgaacgtctgctggagaacattactgttctggcagaagcggcggcgctggcaacgtctccggcgctgacagatgagctggtcagccacggcgagctgatgtcgaccctgctgtttgttgagatcctgcgcgaacgcgatgttcaggcacagtggtttgatgtacgtaaagtgatgcgtaccaacgaccgatttggtcgtgcagagccagatatagccgcgctggcggaactggccgcgctgcagctgctcccacgtctcaatgaaggcttagtgatcacccagggatttatcggtagcgaaaataaaggtcgtacaacgacgcttggccgtggaggcagcgattatacggcagccttgctggcggaggctttacacgcatctcgtgttgatatctggaccgacgtcccgggcatctacaccaccgatccacgcgtagtttccgcagcaaaacgcattgatgaaatcgcgtttgccgaagcggcagagatggcaacttttggtgcaaaagtactgcatccggcaacgttgctacccgcagtacgcagcgatatcccggtctttgtcggctccagcaaagacccacgcgcaggtggtacgctggtgtgcaataaaactgaaaatccgccgctgttccgcgctctggcgcttcgtcgcaatcagactctgctcactttgcacagcctgaatatgctgcattctcgcggtttcctcgcggaagttttcggcatcctcgcgcggcataatatttcggtagacttaatcaccacgtcagaagtgagcgtggcattaacccttgataccaccggttcaacctccactggcgatacgttgctgacgcaatctctgctgatggagctttccgcactgtgtcgggtggaggtggaagaaggtctggcgctggtcgcgttgattggcaatgacctgtcaaaagcctgcggcgttggcaaagaggtattcggcgtactggaaccgttcaacattcgcatgatttgttatggcgcatccagccataacctgtgcttcctggtgcccggcgaagatgccgagcaggtggtgcaaaaactgcatagtaatttgtttgagtaa

SEQ ID NO: 23 - DNA, 1104 bp, Artificial Sequence
  Synthetic DNA
atgaaaaatgttggttttatcggctggcgcggtatggtcggctccgttctcatgcaacgcatggttgaagagcgcgacttcgacgccattcgccctgtcttcttttctacttctcagcttggccaggctgcgccgtcttttggcggaaccactggcacacttcaggatgcctttgatctggaggcgctaaaggccctcgatatcattgtgacctgtcagggcggcgattataccaacgaaatctatccaaagcttcgtgaaagcggatggcaaggttactggattgacgcagcatcgtctctgcgcatgaaagatgacgccatcatcattcttgaccccgtcaatcaggacgtcattaccgacggattaaataatggcatcaggacttttgttggcggtaactgtaccgtaagcctgatgttgatgtcgttgggtggtttattcgccaatgatcttgttgattgggtgtccgttgcaacctaccaggccgcttccggcggtggtgcgcgacatatgcgtgagttattaacccagatgggccatctgtatggccatgtggcagatgaactcgcgaccccgtcctctgctattctcgatatcgaacgcaaagtcacaaccttaacccgtagcggtgagctgccggtggataactttggcgtgccgctggcgggtagcctgattccgtggatcgacaaacagctcgataacggtcagagccgcgaagagtggaaagggcaggcggaaaccaacaagatcctcaacacatcttccgtaattccggtagatggtttatgtgtgcgtgtcggggcattgcgctgccacagccaggcattcactattaaattgaaaaaagatgtgtctattccgaccgtggaagaactgctggctgcgcacaatccgtgggcgaaagtcgttccgaacgatcgggaaatcactatgcgtgagctaaccccagctgccgttaccggcacgctgaccacgccggtaggccgcctgcgtaagctgaatatgggaccagagttcctgtcagcctttaccgtgggcgaccagctgctgtggggggccgcggagccgctgcgtcggatgcttcgtcaactggcgtaa

SEQ ID NO: 24 - DNA, 2391 bp, Artificial Sequence
  Synthetic DNA
atgcgagtgttgaagttcggcggtacatcagtggcaaatgcagaacgttttctgcgtgttgccgatattctggaaagcaatgccaggcaggggcaggtggccaccgtcctctctgcccccgccaaaatcaccaaccacctggtggcgatgattgaaaaaaccattagcggccaggatgctttacccaatatcagcgatgccgaacgtatttttgccgaacttttgacgggactcgccgccgcccagccggggttcccgctggcgcaattgaaaactttcgtcgatcaggaatttgcccaaataaaacatgtcctgcatggcattagtttgttggggcagtgcccggatagcatcaacgctgcgctgatttgccgtggcgagaaaatgtcgatcgccattatggccggcgtattagaagcgcgcggtcacaacgttactgttatcgatccggtcgaaaaactgctggcagtggggcattacctcgaatctaccgtcgatattgctgagtccacccgccgtattgcggcaagccgcattccggctgatcacatggtgctgatggcaggtttcaccgccggtaatgaaaaaggcgaactggtggtgcttggacgcaacggttccgactactctgctgcggtgctggctgcctgtttacgcgccgattgttgcgagatttggacggacgttgacggggtctatacctgcgacccgcgtcaggtgcccgatgcgaggttgttgaagtcgatgtcctaccaggaagcgatggagctttcctacttcggcgctaaagttcttcacccccgcaccattacccccatcgcccagttccagatcccttgcctgattaaaaataccggaaatcctcaagcaccaggtacgctcattggtgccagccgtgatgaagacgaattaccggtcaagggcatttccaatctgaataacatggcaatgttcagcgtttctggtccggggatgaaagggatggtcggcatggcggcgcgcgtctttgcagcgatgtcacgcgcccgtatttccgtggtgctgattacgcaatcatcttccgaatacagcatcagtttctgcgttccacaaagcgactgtgtgcgagctgaacgggcaatgcaggaagagttctacctggaactgaaagaaggcttactggagccgctggcagtgacggaacggctggccattatctcggtggtaggtgatggtatgcgcaccttgcgtgggatctcggcgaaattctttgccgcactggcccgcgccaatatcaacattgtcgccattgctcagggatcttctgaacgctcaatctctgtcgtggtaaataacgatgatgcgaccactggcgtgcgcgttactcatcagatgctgttcaataccgatcaggttatcgaagtgtttgtgattggcgtcggtggcgttggcggtgcgctgctggagcaactgaagcgtcagcaaagctggctgaagaataaacatatcgacttacgtgtctgcggtgttgccaactcgaaggctctgctcaccaatgtacatggccttaatctggaaaactggcaggaagaactggcgcaagccaaagagccgtttaatctcgggcgcttaattcgcctcgtgaaagaatatcatctgctgaacccggtcattgttgactgcacttccagccaggcagtggcggatcaatatgccgacttcctgcgcgaaggtttccacgttgtcacgccgaacaaaaaggccaacacctcgtcgatggattactaccatcagttgcgttatgcggcggaaaaatcgcggcgtaaattcctctatgacaccaacgttggggctggattaccggttattgagaacctgcaaaatctgctcaatgcaggtgatgaattgatgaagttctccggcattctttctggttcgctttcttatatcttcggcaagttagacgaaggcatgagtttctccgaggcgaccacgctggcgcgggaaatgggttataccgaaccggacccgcgagatgatctttctggtatggatgtggcgcgtaaactattgattctcgctcgtgaaacgggacgtgaactggagctggcggatattgaaattgaacctgtgctgcccgcagagtttaacgccgagggtgatgttgccgcttttatggcgaatctgtcacaactcgacgatctctttgccgcgcgcgtggcgaaggcccgtgatgaaggaaaagttttgcgctatgttggcaatattgatgaagatggcgtctgccgcgtgaagattgccgaagtggatggtaatgatccgctgttcaaagtgaaaaatggcgaaaacgccctggccttctatagccactattatcagccgctgccgttggtactgcgcggatatggtgcg

SEQ ID NO: 25 - DNA, 933 bp, Artificial Sequence
  Synthetic DNA
atggttaaagtttatgccccggcttccagtgccaatatgagcgtcgggtttgatgtgctcggggcggcggtgacacctgttgatggtgcattgctcggagatgtagtcacggttgaggcggcagagacattcagtctcaacaacctcggacgctttgccgataagctgccgtcagaaccacgggaaaatatcgtttatcagtgctgggagcgtttttgccaggaactgggtaagcaaattccagtggcgatgaccctggaaaagaatatgccgatcggttcgggcttaggctccagtgcctgttcggtggtcgcggcgctgatggcgatgaatgaacactgcggcaagccgcttaatgacactcgtttgctggctttgatgggcgagctggaaggccgtatctccggcagcattcattacgacaacgtggcaccgtgttttctcggtggtatgcagttgatgatcgaagaaaacgacatcatcagccagcaagtgccagggtttgatgagtggctgtgggtgctggcgtatccggggattaaagtctcgacggcagaagccagggctattttaccggcgcagtatcgccgccaggattgcattgcgcacgggcgacatctggcaggcttcattcacgcctgctattcccgtcagcctgagcttgccgcgaagctgatgaaagatgttatcgctgaaccctaccgtgaacggttactgccaggcttccggcaggcgcggcaggcggtcgcggaaatcggcgcggtagcgagcggtatctccggctccggcccgaccttgttcgctctgtgtgacaagccggaaaccgcccagcgcgttgccgactggttgggtaagaactacctgcaaaatcaggaaggttttgttcatatttgccggctggatacggcgggcgcacgagtactggaaaactaa

SEQ ID NO: 26 - DNA, 1287 bp, Artificial Sequence
  Synthetic DNA
atgaaactctacaatctgaaagatcacaacgagcaggtcagctttgcgcaagccgtaacccaggggttgggcaaaaatcaggggctgttttttccgcacgacctgccggaattcagcctgactgaaattgatgagatgctgaagctggattttgtcacccgcagtgcgaagatcctctcggcgtttattggtgatgaaatcccacaggaaatcctggaagagcgcgtgcgcgcggcgtttgccttcccggctccggtcgccaatgttgaaagcgatgtcggttgtctggaattgttccacgggccaacgctggcatttaaagatttcggcggtcgctttatggcacaaatgctgacccatattgcgggtgataagccagtgaccattctgaccgcgacctccggtgataccggagcggcagtggctcatgctttctacggtttaccgaatgtgaaagtggttatcctctatccacgaggcaaaatcagtccactgcaagaaaaactgttctgtacattgggcggcaatatcgaaactgttgccatcgacggcgatttcgatgcctgtcaggcgctggtgaagcaggcgtttgatgatgaagaactgaaagtggcgctagggttaaactcggctaactcgattaacatcagccgtttgctggcgcagatttgctactactttgaagctgttgcgcagctgccgcaggagacgcgcaaccagctggttgtctcggtgccaagcggaaacttcggcgatttgacggcgggtctgctggcgaagtcactcggtctgccggtgaaacgttttattgctgcgaccaacgtgaacgataccgtgccacgtttcctgcacgacggtcagtggtcacccaaagcgactcaggcgacgttatccaacgcgatggacgtgagtcagccgaacaactggccgcgtgtggaagagttgttccgccgcaaaatctggcaactgaaagagctgggttatgcagccgtggatgatgaaaccacgcaacagacaatgcgtgagttaaaagaactgggctacacttcggagccgcacgctgccgtagcttatcgtgcgctgcgtgatcagttgaatccaggcgaatatggcttgttcctcggcaccgcgcatccggcgaaatttaaagagagcgtggaagcgattctcggtgaaacgttggatctgccaaaagagctggcagaacgtgctgatttacccttgctttcacataatctgcccgccgattttgctgcgttgcgtaaattgatgatgaatcatcagtaa

SEQ ID NO: 27 - DNA, 888 bp, Artificial Sequence
  Synthetic DNA
atgcctggttcattacgtaaaatgccggtctggttaccaatagtcatattgctcgttgccatggcgtctattcagggtggagcctcgttagctaagtcactttttcctctggtgggcgcaccgggtgtcactgcgctgcgtctggcattaggaacgctgatcctcatcgcgttctttaagccatggcgactgcgctttgccaaagagcaacggttaccgctgttgttttacggcgtttcgctgggtgggatgaattatcttttttatctttctattcagacagtaccgctgggtattgcggtggcgctggagttcaccggaccactggcggtggcgctgttctcttctcgtcgcccggtagatttcgtctgggttgtgctggcggttcttggtctgtggttcctgctaccgctggggcaagacgtttcccatgtcgatttaaccggctgtgcgctggcactgggggccggggcttgttgggctatttacattttaagtgggcaacgcgcaggagcggaacatggccctgcgacggtggcaattggttcgttgattgcagcgttaattttcgtgccaattggagcgcttcaggctggtgaagcactctggcactggtcggttattccattgggtctggctgtcgctattctctcgaccgctctgccttattcgctggaaatgattgccctcacccgtttgccaacacggacatttggtacgctgatgagcatggaaccggcgctggctgccgtttccgggatgattttcctcggagaaacactgacacccatacagctactggcgctcggcgctatcatcgccgcttcaatggggtctacgctgacagtacgcaaagagagcaaaataaaagaattagacattaattaa

SEQ ID NO: 28 - DNA, 1719 bp, Artificial Sequence
  Synthetic DNA
atgaaacaaacggttgcagcttatatcgccaaaacactcgaatcggcaggggtgaaacgcatctggggagtcacaggcgactctctgaacggtcttagtgacagtcttaatcgcatgggcaccatcgagtggatgtccacccgccacgaagaagtggcggcctttgccgctggcgctgaagcacaacttagcggagaactggcggtctgcgccggatcgtgcggccccggcaacctgcacttaatcaacggcctgttcgattgccaccgcaatcacgttccggtactggcgattgccgctcatattccctccagcgaaattggcagcggctatttccaggaaacccacccacaagagctattccgcgaatgtagtcactattgcgagctggtttccagcccggagcagatcccacaagtactggcgattgccatgcgcaaagcggtgcttaaccgtggcgtttcggttgtcgtgttaccaggcgacgtggcgttaaaacctgcgccagaaggggcaaccatgcactggtatcatgcgccacaaccagtcgtgacgccggaagaagaagagttacgcaaactggcgcaactgctgcgttattccagcaatatcgccctgatgtgtggcagcggctgcgcgggggcgcataaagagttagttgagtttgccgggaaaattaaagcgcctattgttcatgccctgcgcggtaaagaacatgtcgaatacgataatccgtatgatgttggaatgaccgggttaatcggcttctcgtcaggtttccataccatgatgaacgccgacacgttagtgctactcggcacgcaatttccctaccgcgccttctacccgaccgatgccaaaatcattcagattgatatcaacccagccagcatcggcgctcacagcaaggtggatatggcactggtcggcgatatcaagtcgactctgcgtgcattgcttccattggtggaagaaaaagccgatcgcaagtttctggataaagcgctggaagattaccgcgacgcccgcaaagggctggacgatttagctaaaccgagcgagaaagccattcacccgcaatatctggcgcagcaaattagtcattttgccgccgatgacgctattttcacctgtgacgttggtacgccaacggtgtgggcggcacgttatctaaaaatgaacggcaagcgtcgcctgttaggttcgtttaaccacggttcgatggctaacgccatgccgcaggcgctgggtgcgcaggcgacagagccagaacgtcaggtggtcgccatgtgcggcgatggcggttttagcatgttgatgggcgatttcctctcagtagtgcagatgaaactgccagtgaaaattgtcgtctttaacaacagcgtgctgggctttgtggcgatggagatgaaagctggtggctatttgactgacggcaccgaactacacgacacaaactttgcccgcattgccgaagcgtgcggcattacgggtatccgtgtagaaaaagcgtctgaagttgatgaagccctgcaacgcgccttctccatcgacggtccggtgttggtggatgtggtggtcgccaaagaagagttagccattccaccgcagatcaaactcgaacaggccaaaggtttcagcctgtatatgctgcgcgcaatcatcagcggacgcggtgatgaagtgatcgaactggcgaaaacaaactggctaaggtaa

SEQ ID NO: 29 - DNA, 2283 bp, Artificial Sequence
  Synthetic DNA
atgtccgagcttaatgaaaagttagccacagcctgggaaggttttaccaaaggtgactggcagaatgaagtaaacgtccgtgacttcattcagaaaaactacactccgtacgagggtgacgagtccttcctggctggcgctactgaagcgaccaccaccctgtgggacaaagtaatggaaggcgttaaactggaaaaccgcactcacgcgccagttgactttgacaccgctgttgcttccaccatcacctctcacgacgctggctacatcaacaagcagcttgagaaaatcgttggtctgcagactgaagctccgctgaaacgtgctcttatcccgttcggtggtatcaaaatgatcgaaggttcctgcaaagcgtacaaccgcgaactggatccgatgatcaaaaaaatcttcactgaataccgtaaaactcacaaccagggcgtgttcgacgtttacactccggacatcctgcgttgccgtaaatctggtgttctgaccggtctgccagatgcatatggccgtggccgtatcatcggtgactaccgtcgcgttgcgctgtacggtatcgactacctgatgaaagacaaactggcacagttcacttctctgcaggctgatctggaaaacggcgtaaacctggaacagactatccgtctgcgcgaagaaatcgctgaacagcaccgcgctctgggtcagatgaaagaaatggctgcgaaatacggctacgacatctctggtccggctaccaacgctcaggaagctatccagtggacttacttcggctacctggctgctgttaagtctcagaacggtgctgcaatgtccttcggtcgtacctccaccttcctggatgtgtacatcgaacgtgacctgaaagctggcaagatcaccgaacaagaagcgcaggaaatggttgaccacctggtcatgaaactgcgtatggttcgcttcctgcgtactccggaatacgatgaactgttctctggcgacccgatctgggcaaccgaatctatcggtggtatgggcctcgacggtcgtaccctggttaccaaaaacagcttccgtttcctgaacaccctgtacaccatgggtccgtctccggaaccgaacatgaccattctgtggtctgaaaaactgccgctgaacttcaagaaattcgccgctaaagtgtccatcgacacctcttctctgcagtatgagaacgatgacctgatgcgtccggacttcaacaacgatgactacgctattgcttgctgcgtaagcccgatgatcgttggtaaacaaatgcagttcttcggtgcgcgtgcaaacctggcgaaaaccatgctgtacgcaatcaacggcggcgttgacgaaaaactgaaaatgcaggttggtccgaagtctgaaccgatcaaaggcgatgtcctgaactatgatgaagtgatggagcgcatggatcacttcatggactggctggctaaacagtacatcactgcactgaacatcatccactacatgcacgacaagtacagctacgaagcctctctgatggcgctgcacgaccgtgacgttatccgcaccatggcgtgtggtatcgctggtctgtccgttgctgctgactccctgtctgcaatcaaatatgcgaaagttaaaccgattcgtgacgaagacggtctggctatcgacttcgaaatcgaaggcgaatacccgcagtttggtaacaatgatccgcgtgtagatgacctggctgttgacctggtagaacgtttcatgaagaaaattcagaaactgcacacctaccgtgacgctatcccgactcagtctgttctgaccatcacttctaacgttgtgtatggtaagaaaacgggtaacaccccagacggtcgtcgtgctggcgcgccgttcggaccgggtgctaacccgatgcacggtcgtgaccagaaaggtgcagtagcctctctgacttccgttgctaaactgccgtttgcttacgctaaagatggtatctcctacaccttctctatcgttccgaacgcactgggtaaagacgacgaagttcgtaagaccaacctggctggtctgatggatggttacttccaccacgaagcatccatcgaaggtggtcagcacctgaacgttaacgtgatgaaccgtgaaatgctgctcgacgcgatggaaaacccggaaaaatatccgcagctgaccatccgtgtatctggctacgcagtacgtttcaactcgctgactaaagaacagcagcaggacgttattactcgtaccttcactcaatctatgtaa

SEQ ID NO: 30 - DNA, 13863 bp, Artificial Sequence
  Synthetic DNA
gtgctgcagccgctgccgccgattccggatctcattgccacgcgcccccgacgaccgcccgacgtgcattcccggtacggtagggccctgcgcgcacggcgccagagggatgggcgggtagagccaactgcctctggttctgctggcctccgctgctcgcgaagggattcctgctcccgggaggtgtaggagccgctttccagaagcacagcccagagacgtctgggcggcggcccacacaacgcatgtgttcggagctcgccgcgctctgcttttgctctaagcgggaaccatggcttctggccacgctggggaaccgaggaggtggccgcacccaagcaggggtcgaaagcccgggtggatgcggaacaaggatatgataggccttaagggtgggggatacctctgggctcgaaatcggcgggcggtgcaaaactcgaggtccagttctcggagcccatagagccaaaaaagcctcagcttgtccggggcgggttcttgaaagacggaaagcggctgagtaccacgcggcttgcatttttctcttgggacgctcgagaggtgggctccgtgagggcagctgctgcctgcagattatagggagccctttgcgcatttattaagaagctactggtgtatctcgggctgcgctaggcacggcgcatgcaaagatgaagcaggcagcatcccagcccttccgcacctcagacggtcagttgagtaggatccgccggtaccaactcctccttttaacaaatagggagaccgaaagctaggagacagtcagggatctctaagttcccagtgagtaggaggcagaggtgaggtgtagaactcgtttttgcatgtctctcgcctctagacgcacccttccctcatcccatgccctcccacctccgcccctacattaaaggtagcattggatcccggggccgttcagtgaagctagcaggtgtccgcaggaactcccttccccctgccaggctagaaaccttacaaggctgtctagaaatagcagtgatttgtaaggagagacccggctccagcttggtgactctgggctgactgcctgcctagaggtcctctcggatttttgccctttggagtggtgtcaaaactagacgtgatactttggggatgcagcctgtgatatttcctccagcgaatgcagtgcagggttggattaacaaggtggaaagaattcgagggttccaccaagtagctattaactctagggctgcaggcctcaggccttctgcagctatttctacactccctgtactgaaactatttcttcatactgggcctgacaggcctttgcaacaaggatcacggccgaagccacaccgtgcgcctccctcccggttggttaacaggccctggtttctagtattgcgatttaaagtctggcgctggctgcgcgccagacctgggaggctgccagctaggcttcacgttgctggcgtctgcttcggggcattcattaggtctgaagtctgaatcccagctccctccctctcacccactgagctgcatagctccagattgcctctgcttacgggcggggcttctcagccttctgccttctggcccgatgcccgcttcccaacggccggaggccgctagactaatcggcttcgccctgcgcgctgtaatgcgcatgcgcacgcgcacaagttcctgggcccgcccatcttccggacttgggcggggcgtaaaagccgggcgttcggaggacccagcaattagtctgatttccgcccacctttccgagcgggaaggagagccacaaagcgcgcatgcgcgcggatcaccgcaggctcctgtgccttgggcttgagctttgtggcagttaatggcttttctgcacgtatctctggtgtttacttgagaagcctggctgtgtccttgctgtaggagccggagtagctcagagtgatcttgtctgaggaaaggccagccccacttggggttaataaaccgcgatgggtgaaccctcaggaggctatacttacacccaaacgtcgatattccttttccacgctaaggtatgggccttcactcttcacagaccctgtcattaggcctttcaactctcttttggcaaccattaggttttttcccctccctttttagtcatctctagtgatttatagtggcaaatacccccaaaggaagtaaaatagcttaaaaaaatctcttggttaataaacattaaagaagctgtagtgacactaaatgtttttcctcctatagattccttttggttccaagtccaatatggcaactctaaaggatcagctgatttataatcttctaaaggaagaacagaccccccagaataagattacagttgttggggttggtgctgttggcatggcctgtgccatcagtatcttaatgaaggtaagtgagagtctaccacactggaagcccataccttgaccccatcctctacccccactcctacccctagaactgtattattacatttcatgtaacagtatttagatttatgcactcattcggataactttctgtgaaacaaacttttgaaatatgataatacaccaaaagtgtatctgaaattaaaaagaatcaaaggttgtcaggctggagacccagttcctaaaattcattattctgtattaacatgcatggattgactaccaatgaaaaggaagggtccatgattttaaatgagccaaaattcttttaaagtgatttttgaattgaaaatgacaattcaaaaattgtcatttattggtaaaattatatgggaaatcataagttctcccactcaaatctcattgcccctgtgccttggatagcaattttgttatcaattatggagctaaaatttaattagaaaaaagaaattgtgagtaaagcactccttattacactattgaaagctgatttatatttaaaagaaattgaggcagcttacaacattaaaatgtctgaggcggggcacagtggctcatgcttgtaatgccagcactttaggaggctgaggtgggtggatcacgaggtcaggagatggagaccatcctggctaacacgatgaaaccccatctttactagaaatacaaaaaattagccgggcgtggtggcatacgcctatagtcccagctacttgggaggctgaggcaggagaattgcttgaacccaggaggtggaggtggcagtgacccgagatagcaccactccactccagcctgggcgacagtgagactccatctcaaaaaaaaaaatctgaagttaagatgtggagtgtctaataaaagtaaaatgatgaattctgggttctaaatagaaatggattcaagtgagaagggactaaagacagaaatgagctatgaaaaggcctcgtaacaacacaggtgactctacatatgttcttaggaaaggccacataatacaccaacttttattccttacccactagatgagaaattgatgctgttttccccacacctacaaaccgcctatgttttttctctgtgatggcctctggctcaggtgtgggtaagaagagtaactgacactcattatattgtggatgatttagggatagatctgcagcttgaataacttttggtaacgatagaccacatccagttgtattaaagctgttattggtgctcctggcctgaaatggacctatgaactttgagttgcaactataaggatattttttgccagtattatacactgcacaaacctatttatccataactgttagtattggttcatatatggaatcaaccagggaatagttcagattccatctctgaaagatgggcggaaatcagactttttaactttttaagttttttttttttgagacggaatctcgctttgttgccctggctggagtgcagtggcacgatcttggctcacttgacctcctgggttcaagtgattctcctgcctcagcctcccgagtagctgggactacaggcacccaccgccacgcctggctgatttttgtatttttagtagagacaggccttcaccatattggccaggctggtcttttttttttttttttttttttttttctgagaaggagtctcgccgtgtcgcccaggctagagtgcagtggcgtgaactccgctcactgctagctctgcctcccgggttcataccattctcctgtctcagcctcccaagtagctgggactacaggcacccaccaccacgcctggctaaatgtttgtattttttagtagagacggggtttcaccatgttagccaggatggtctcgatctcctgacctcgtgatccgcctaccttggcctcccaaagtgctgggattacaagcgtgagccaccgtgcctggcctggccaggctgtcttgaactcctgacctcaagtgatgtgcccgcctcggcctcccaaagtgttgggattacagatgtgagtcactatgcccggccagaacatttcttactaatttcaagtcttgatgctggtcaatatcacctagttaaatgaataacaacctaaaattggtgtgtaggatggaatttgagagagtagacagagcagttttatataattggaagttattctagcaactgccagtccagtgttctgcttccacatctgcagtggtggaactcctatagagctcgcttcagtggggagacagggctggagagagggtcagtgctatctatgtagggtgtaatctgtaagtcagcttttgaaatggggtgccctctactttgaatatctcgatactgtactaataaagtaacagaactctcctatgccagaaatatagaaatttttcatgctcttctaaaaatctagaagtggcaattttccatttaactaaagatttgatgtcttttaggacttggcagatgaacttgctcttgttgatgtcatcgaagacaaattgaagggagagatgatggatctccaacatggcagccttttccttagaacaccaaagattgtctctggcaaaggttgatttcaacaagtttatattataatccatgcttgacttaaattctttttccagatggtctccatttgttgcttagggtagagtgcagttgcacaattatggctcaccacagcctcgaaccctgggctcaagcaatcctccttccacttcattaccccctccccctcacaaagaaactgggactatagggtatgctaccatgcccggctaatttttttactttttgtagagatggggacccactgtgttgcccaggcctgtcttgaaccactgggctcaagtgatcctccctccttagccttccgaagtactgggattgcaggtgtgaaccactgtgcccggctttagacttaaatgttttatcaggcttgaaatcctagctctttaaagattttgttttaaatgccgggtgcaagagcctgggaacaatttcacttaggtgcctgtgaatatcaaagtttcaatttctggcaaatggtttaaaatagaaatccaatttgtccatgctatgcaaaccatctgaattagaatgtaatgagtaaagcttaaaccttaggtctgtatttaaccacattgtgttacttacttgcccccacatcctttcacacacgaagttgagaatagggtaaataaatgagcctgttcagctaatactcttggcttgaccctttcacacttaacagcaccagccaagaaacctgaatgtgagcccaaatagtgtctattttgatacctgaaaatcactggccaccttgctgatgggcaactcccttcatcactggtttaactctcttgtgccatagggtatctagaagcaaaatatgtttgttaagtgtaaagctgtctctgcttaaaaacaagtccccctaccaccaccaccacacacacacacacacacacacacacacacacacacacacacacacacacacgaaattgcctgttcctgggctgataggacaccagttaagtagaaacaggagtatggaagagtgtgaacgttgagcttggggatcaaaaatttgaggatatgtaagaaattaataggagaatcaaataataaacttgatttcctccagctctccctaattgtagttacataaagttacaacttgactaaaactacaaggaagatgttgacatgctcttcctccatttaagaagccataatgataaaactctaagaacaagaaaggtttgtggagcatttatggaacaaatttttgctgcctaggtaaaatttattctaaaggccttaatctggtcattattccccttttctctagactataatgtaactgcaaactccaagctggtcattatcacggctggggcacgtcagcaagagggagaaagccgtcttaatttggtccagcgtaacgtgaacatctttaaattcatcattcctaatgttgtaaaatacagcccgaactgcaagttgcttattgtttcaaatccaggtgaggcttttgactgcataaaaattgacaagctatagtaaaactgatagtatatgatatatatattatatatattttaaatattttgaaatattttaaaaaatacatttttaaaaatattttcgaatattattttaaaatatatatatatattttgaggcggagttttgctcttgtcgcccaggttggagtgcagtggcgcaatctgggctcactgcaacctctgcctcatgggttcaagcgattcttttgcctcagcctctcaagtagctgggattataagcgcctgccaccacacatggctaattttttatatttttagtagagacagggtttcaccatgttggccaggctggttttgaactcctggcctcaagcagtccatctgcctcccaaagtgctaggattacaggcgtgagccaccgtgcccagccacgcatatttattgattcatttatttttcttttttttttttttttttttttgagacggagtcttgctctgtcaccctggctggagtacagtggcttgatcttggctcactgcaagctccgcctcccgggttcatgccattctcctgcctcagcctcccgagtagctgggactacaggtgcccaccacgacgcctggctaattttttgtatttttagtagagacggggtttcatcaggttagccaggatggtctcgatctcctgacctcgtgatctgcccgccttggcctcccaaagtgctgggattacaggcgtgagccaccgtgcctggtgattcatttatttttcatgtttcatttcccttctaaggagatttgtgtgtgtgtgttttttgttttttaataattttaaaacattaaagggaatacaatgcctttaaatgtagttggagcttaaaattacctgcccaagatcttggataagggataagtttgtgaataattgttattctctttttttttttttttttttttgagacagtctcactttgtagctcaggctggagtgcagtggttcgatcttggctcactgcaacctctgcctcctgggttcaagcaattctcctgcctcagcctcccaagagctgggattacaggcacgtgccaccatgctcggctaatttttgaagttttagtagaaaggggtttcaccatgttgcccaggctggtctcaaattcctgagctcaggtgatccatctgcctcagcctcccaaagtattaggattacaggcgtgagccaccgtgcccgggcccataattgtctcttagttgataaacagtttattttcataaaactgttactatactttttttttgagagcatgtctcactctgtcgcccaagctggagggcaatgggatgatcatggcagctttgacctactaggctcaggtgatccttcttcctcagcctcttaagtagctaggactacaggcgtgcaccaatatgcctggctagtttgttaaaagtttttttgtagagatggggttttgctatgttgcccaggctggtcttgaactgctggcctcaggcagtcctcccacctcagcctcccaaagtgttgggataacaggtgtgagttgtcatgcccagccaaaactactttttgaataattaatggacttgatatacatagtgtagaggcttaaaaatattaacaaaattattggttagccatgatcaatatcaagatcctgaaaagccatatatctggagtagcctattattatctaatgatcacctagtatctggttaagtgttttcttcatagtaggtatatcttttttgtgtgtagggagaggataatgggtgatttttattttctcctttttcatagtggatatcttgacctacgtggcttggaagataagtggttttcccaaaaaccgtgttattggaagcggttgcaatctggattcagcccgattccgttacctaatgggggaaaggctgggagttcacccattaagctgtcatgggtgggtccttggggaacatggagattccagtggtaagcataagttattttctttttgtttttgaaaagattatataaaaagtcgatgggcattatattattcaattagagcctaatcaaatatccattcagtaggatggaatggtttcccgaaatctagcattttgtataattatatgttaagaattgttaagattgttgccattttatatggcattttatggcgagggggacgggaaatgaaatttctcttcttaccatggatatcttaagactgtagttcttaggatgtcttcagtcatttaatatcacagctgtttatacctgacttgtactgcctggccctgaaaagatgagcaaatccaaatgcacaaaagttatattatcacagttgaaaaatgttatgattaggttctgtatgctaagaaaaccccccttatgttctcatactatctttatatttcaaatatacatgggttaaacatttcaattggctagagaaacaggttagaatacagttaaaattcttagttttacataatgtaagtaaatgaaaatctaatctaaaagtgagtaatgactacattagtagtcttgaccatctaccaaaattgagtattcttcctccgaagataagagaattaggaaaatgaatcacaattactaatctgttggtacatgaaaataaatgtagtctgtactatttcttttagtgcctgtatggagtggaatgaatgttgctggtgtctctctgaagactctgcacccagatttagggactgataaagataaggaacagtggaaagaggttcacaagcaggtggttgagaggtaataaatctttcaatttggcaacacagaatattaacatttactatttttatttaaaaggttaaaattgtaatagtatttgcatttgagaactttttgttagaaaacttgtgtggtttttttgttttgttttgtttgagacagaatcttgccctttcgcacaggctgcagtgcagtggcgcaatcttggctcactgcaacctctgcctcccgggttcaggcgattctcctgcctcagcctcctgggtacctgggactacaggcatatgccatgacgcccggctaattttttgtatttttagtagagatggggtttcaccatgttagccaaaaaaaaaagaatgtgcctcaccttgcaaggcccaggccctaggatcacttgagctcaggagttcaaggccagcctgggcaacagggcaaaaccctgtctctacaataaatacacaaattagccaggcatggtggtgagcacctgtggtcctagctacttgagaggctgaggcaggaggatcgcatgagcctgggaggtcaaggctgcagtgaagcgagatcctgccactgcactccagagcctgctagcctgggtgacagagtaagagcctgtctcaaaggaaaaaaaaaattattgaaatagggaagctttcaacttggtggcattatttacctttgtggtcctgtgtggacctcaggtctatagaattaaaaaatgaatcatagccgggcatggtggctcatgcctgtaatcccagcactttgggaggccgaggcaggcagatcacgaggtcaggagatggagaccatcctggctaacacggtgaaaccccgtctctactaaaaatacaaaaaattagccgggcgtggtggcaggcgcctgtagtcccagctactcgggaggctgaggcaggagaatggcatgaacccggtagttggagcttgcagtgagccgagatcgcgccactgcactccagcctgggcgacagagcgagactccgtctcaaaaaaaaaagaatcataatctttagttcataacatattcttgtgattggtcaagcaaggccctcttgtttgtatttgtttaattaaataaaacctgtgaacccaccacccagctcaagaaagaaacacaatatctgtcaaataacattgttgaatcagaatttagtattctgctggtgtttggaaataagtggattctgtgctctttcccccagctatccctctgtccccctcacgctcccacttgagataatcctgagttaaggatgctatgttatcttggatttctttttaaaattcaatattatatttttaagaattatccaattttttttacaagtagctatagtttattttttgatagctgtgtaatattccattgtatcagtataccatgatttatccattcttctgttggaggacattggaaagattgtcatgtttttgctgttactaacagtactgttaatgaatatccctgtacataatatcctagcatacatgtgtgcaagggttattcttggtataatgcaacattgtggcattatttactgtaaaatgtgtattaatgaaaactttgtttttctttctttctcccaccctgctttttctgcctttacctatggtttcctatcatacagtgcttatgaggtgatcaaactcaaaggctacacatcctgggctattggactctctgtagcagatttggcagagagtataatgaagaatcttaggcgggtgcacccagtttccaccatgattaaggtaggtctatgtagtgatacgctgcatttgaatgctttttgctggctttttaaaaaagattcttctgagaaagattaatacaagtcttccattactgacttaagtgaaataaattaatgtacccacagcttaccttttttgaaagaaatggttgagctttaggattaatgtccattaggcctgttcaacacatagatacttgataatttgactacaaaaaagtcttgttcaattatgctgaggtaggtggaagactataaaagaaataaactatttctccattggggaaaatagaaattatattcaagttagcattatgttactatttttaatgactttcttttatactattaattaaatcataactgaacacctggaaaggaatttctacttatcaaagttttttatttttttgagacagtctccctctgtcacccaggctgcagtgcagtggccgatctcggctcaccgcaacctctgcctcccaggtttaagcgattcttctgcctcagcctcctaagtagctgggactacaggtgcgtgccaccacgcccggctactttttgtatttttagtagagatggagtttcaccatattggctaggctggtctcgaactcctgaccttgtgatccacccgcctcggcctccccgaatgctgggattgcaggtgtgagccaccgcacctggcctcaagttgtattttaaaatcttcataattaggccacacacagtgactgacagctgtaatgccagcactttggaaggccaagggcaggagaattgcttgagcccaggtgtttgagaccaccctaggcagtatagtgagatcttgcctctgttaaaaaaaaaaaaaaaaaaaaaggccatgtgcgggcagctgatgcctgtaatcccagcactttgggaggccaaggggtggatcacctgaggtcagtagttcaagaccagcctgaccaacatggtgaaaccctgtctctactaaaaatacagaattagccaggtgtggtggcaggcgcctgtaatcccagctacttgggagactgaggcagaagaatcacttgaacccaggaggtggaggttgcagtgagctgagatcgcaccattgcactccagcctgggcaacaagagtgaaactccatctcaaaagaaaaaaaaaagcggctgggctctgtggctcatgcttgtaaccccagcactttgggaggccaagaggtggatcacctgaggtcaagaatttgagaccaacctggccaacatggtgaaaccccatctgtactaaacatacaaaaattagccaagtgtggtggcgcacgcctgtagtcccagaaggctgaagcaggagaattacttgaaccctggaggtggaggttgcggtgagctgagatcgtgccactgcactccagcctgggcgacagagcgagactctgcctcaaaaaaaaattaaaaaaaaaaagctttataattatagagactgtaagtcttgggaaacctgggaatgcatagacaaaatgtgagattttttttttttcatttcatcttcagggtctttacggaataaaggatgatgtcttccttagtgttccttgcattttgggacagaatggaatctcagaccttgtgaaggtgactctgacttctgaggaagaggcccgtttgaagaagagtgcagatacactttgggggatccaaaaggagctgcaattttaaagtcttctgatgtcatatcatttcactgtctaggctacaacaggattctaggtggaggttgtgcatgttgtcctttttatctgatctgtgattaaagcagtaatattttaagatggactgggaaaaacatcaactcctgaagttagaaataagaatggtttgtaaaatccacagctatatcctgatgctggatggtattaatcttgtgtagtcttcaactggttagtgtgaaatagttctgccacctctgacgcaccactgccaatgctgtacgtactgcatttgccccttgagccaggtggatgtttaccgtgtgttatataacttcctggctccttcactgaacatgcctagtccaacattttttcccagtgagtcacatcctgggatccagtgtataaatccaatatcatgtcttgtgcataattcttccaaaggatcttattttgtgaactatatcagtagtgtacattaccatataatgtaaaaagatctacatacaaacaatgcaaccaactatccaagtgttataccaactaaaacccccaataaaccttgaacagtgactactttggttaattcattatattaagatataaagtcataaagctgctagttattatattaatttggaaatattaggctattcttgggcaaccctgcaacgattttttctaacagggatattattgactaatagcagaggatgtaatagtcaactgagttgtattggtaccacttccattgtaagtcccaaagtattatatatttgataataatgctaatcataattggaaagtaacattctatatgtaaatgtaaaatttatttgccaactgaatataggcaatgatagtgtgtcactatagggaacacagatttttgagatcttgtcctctggaagctggtaacaattaaaaacaatcttaaggcagggtgcagtggctcatgcctataatcccagcactttgggaagcccaggtgggctgatcactggaggccaggaattggggaccagcctggccaacacaacaaaaccccatctgttaaaaaaacaaaacaaaaccaaaaaaaacaagtaaccttggtggatgtctactcaagttttctgcacatttttctgaaaatacaactgtgaccctta

SEQ ID NO: 31 - DNA, 2676 bp, Artificial Sequence
  Synthetic DNA
atggctgttactaatgtcgctgaacttaacgcactcgtagagcgtgtaaaaaaagcccagcgtgaatatgccagtttcactcaagagcaagtagacaaaatcttccgcgccgccgctctggctgctgcagatgctcgaatcccactcgcgaaaatggccgttgccgaatccggcatgggtatcgtcgaagataaagtgatcaaaaaccactttgcttctgaatatatctacaacgcctataaagatgaaaaaacctgtggtgttctgtctgaagacgacacttttggtaccatcactatcgctgaaccaatcggtattatttgcggtatcgttccgaccactaacccgacttcaactgctatcttcaaatcgctgatcagtctgaagacccgtaacgccattatcttctccccgcacccgcgtgcaaaagatgccaccaacaaagcggctgatatcgttctgcaggctgctatcgctgccggtgctccgaaagatctgatcggctggatcgatcaaccttctgttgaactgtctaacgcactgatgcaccacccagacatcaacctgatcctcgcgactggtggtccgggcatggttaaagccgcatacagctccggtaaaccagctatcggtgtaggcgcgggcaacactccagttgttatcgatgaaactgctgatatcaaacgtgcagttgcatctgtactgatgtccaaaaccttcgacaacggcgtaatctgtgcttctgaacagtctgttgttgttgttgactctgtttatgacgctgtacgtgaacgttttgcaacccacggcggctatctgttgcagggtaaagagctgaaagctgttcaggatgttatcctgaaaaacggtgcgctgaacgcggctatcgttggtcagccagcctataaaattgctgaactggcaggcttctctgtaccagaaaacaccaagattctgatcggtgaagtgaccgttgttgatgaaagcgaaccgttcgcacatgaaaaactgtccccgactctggcaatgtaccgcgctaaagatttcgaagacgcggtagaaaaagcagagaaactggttgctatgggcggtatcggtcatacctcttgcctgtacactgaccaggataaccaaccggctcgcgtttcttacttcggtcagaaaatgaaaacggcgcgtatcctgattaacaccccagcgtctcagggtggtatcggtgacctgtataacttcaaactcgcaccttccctgactctgggttgtggttcttggggtggtaactccatctctgaaaacgttggtccgaaacacctgatcaacaagaaaaccgttgctaagcgagctgaaaacatgttgtggcacaaacttccgaaatctatctacttccgccgtggctccctgccaatcgcgctggatgaagtgattactgatggccacaaacgtgcgctcatcgtgactgaccgcttcctgttcaacaatggttatgctgatcagatcacttccgtactgaaagcagcaggcgttgaaactgaagtcttcttcgaagtagaagcggacccgaccctgagcatcgttcgtaaaggtgcagaactggcaaactccttcaaaccagacgtgattatcgcgctgggtggtggttccccgatggacgccgcgaagatcatgtgggttatgtacgaacatccggaaactcacttcgaagagctggcgctgcgctttatggatatccgtaaacgtatctacaagttcccgaaaatgggcgtgaaagcgaaaatgatcgctgtcaccaccacttctggtacaggttctgaagtcactccgtttgcggttgtaactgacgacgctactggtcagaaatatccgctggcagactatgcgctgactccggatatggcgattgtcgacgccaacctggttatggacatgccgaagtccctgtgtgctttcggtggtctggacgcagtaactcacgccatggaagcttatgtttctgtactggcatctgagttctctgatggtcaggctctgcaggcactgaaactgctgaaagaatatctgccagcgtcctaccacgaagggtctaaaaatccggtagcgcgtgaacgtgttcacagtgcagcgactatcgcgggtatcgcgtttgcgaacgccttcctgggtgtatgtcactcaatggcgcacaaactgggttcccagttccatattccgcacggtctggcaaacgccctgctgatttgtaacgttattcgctacaatgcgaacgacaacccgaccaagcagactgcattcagccagtatgaccgtccgcaggctcgccgtcgttatgctgaaattgccgaccacttgggtctgagcgcaccgggcgaccgtactgctgctaagatcgagaaactgctggcatggctggaaacgctgaaagctgaactgggtattccgaaatctatccgtgaagctggcgttcaggaagcagacttcctggcgaacgtggataaactgtctgaagatgcattcgatgaccagtgcaccggcgctaacccgcgttacccgctgatctccgagctgaaacagattctgctggatacctactacggtcgtgattatgtagaaggtgaaactgcagcgaagaaagaagctgctccggctaaagctgagaaaaaagcgaaaaaatccgcttaa

SEQ ID NO: 32 - DNA, 2145 bp, Artificial Sequence
  Synthetic DNA
gtgtcccgtattattatgctgatccctaccggaaccagcgtcggtctgaccagcgtcagccttggcgtgatccgtgcaatggaacgcaaaggcgttcgtctgagcgttttcaaacctatcgctcagccgcgtaccggtggcgatgcgcccgatcagactacgactatcgtgcgtgcgaactcttccaccacgacggccgctgaaccgctgaaaatgagctacgttgaaggtctgctttccagcaatcagaaagatgtgctgatggaagagatcgtcgcaaactaccacgctaacaccaaagacgctgaagtcgttctggttgaaggtctggtcccgacacgtaagcaccagtttgcccagtctctgaactacgaaatcgctaaaacgctgaatgcggaaatcgtcttcgttatgtctcagggcactgacaccccggaacagctgaaagagcgtatcgaactgacccgcaacagcttcggcggtgccaaaaacaccaacatcaccggcgttatcgttaacaaactgaacgcaccggttgatgaacagggtcgtactcgcccggatctgtccgagattttcgacgactcttccaaagctaaagtaaacaatgttgatccggcgaagctgcaagaatccagcccgctgccggttctcggcgctgtgccgtggagctttgacctgatcgcgactcgtgcgatcgatatggctcgccacctgaatgcgaccatcatcaacgaaggcgacatcaatactcgccgcgttaaatccgtcactttctgcgcacgcagcattccgcacatgctggagcacttccgtgccggttctctgctggtgacttccgcagaccgtcctgacgtgctggtggccgcttgcctggcagccatgaacggcgtagaaatcggtgccctgctgctgactggcggttacgaaatggacgcgcgcatttctaaactgtgcgaacgtgctttcgctaccggcctgccggtatttatggtgaacaccaacacctggcagacctctctgagcctgcagagcttcaacctggaagttccggttgacgatcacgaacgtatcgagaaagttcaggaatacgttgctaactacatcaacgctgactggatcgaatctctgactgccacttctgagcgcagccgtcgtctgtctccgcctgcgttccgttatcagctgactgaacttgcgcgcaaagcgggcaaacgtatcgtactgccggaaggtgacgaaccgcgtaccgttaaagcagccgctatctgtgctgaacgtggtatcgcaacttgcgtactgctgggtaatccggcagagatcaaccgtgttgcagcgtctcagggtgtagaactgggtgcagggattgaaatcgttgatccagaagtggttcgcgaaagctatgttggtcgtctggtcgaactgcgtaagaacaaaggcatgaccgaaaccgttgcccgcgaacagctggaagacaacgtggtgctcggtacgctgatgctggaacaggatgaagttgatggtctggtttccggtgctgttcacactaccgcaaacaccatccgtccgccgctgcagctgatcaaaactgcaccgggcagctccctggtatcttccgtgttcttcatgctgctgccggaacaggtttacgtttacggtgactgtgcgatcaacccggatccgaccgctgaacagctggcagaaatcgcgattcagtccgctgattccgctgcggccttcggtatcgaaccgcgcgttgctatgctctcctactccaccggtacttctggtgcaggtagcgacgtagaaaaagttcgcgaagcaactcgtctggcgcaggaaaaacgtcctgacctgatgatcgacggtccgctgcagtacgacgctgcggtaatggctgacgttgcgaaatccaaagcgccgaactctccggttgcaggtcgcgctaccgtgttcatcttcccggatctgaacaccggtaacaccacctacaaagcggtacagcgttctgccgacctgatctccatcgggccgatgctgcagggtatgcgcaagccggttaacgacctgtcccgtggcgcactggttgacgatatcgtctacaccatcgcgctgactgcgattcagtctgcacagcagcagtaa

SEQ ID NO: 33 - DNA, 1307 bp, Artificial Sequence
  Synthetic DNA
atgagtacttcagatagcattgtatccagccagacaaaacaatcgtcctggcgtaaatcagataccacatggacgttaggcttgtttggtacggcaatcggcgccggggtgctgttcttccctatccgcgcaggttttggcggactgatcccgattcttctgatgttggtattggcataccccatcgcgttttattgccaccgggcgctggcgcgtctgtgtctttctggctctaacccttccggcaacattacggaaacggtggaagagcattttggtaaaactggcggcgtggttatcacgttcctgtacttcttcgcgatttgcccactgctgtggatttatggcgttactattaccaatacctttatgacgttctgggaaaaccagctcggctttgcaccgctgaatcgcggctttgtggcgctgttcctgttgctgctgatggctttcgtcatctggtttggtaaggatctgatggttaaagtgatgagctacctggtatggccgtttatcgccagcctggtgctgatttctttgtcgctgatcccttactggaactctgcagttatcgaccaggttgacctcggttcgctgtcgttaaccggtcatgacggtatcctgatcactgtctggctggggatttccatcatggttttctcctttaacttctcgccaatcgtctcttccttcgtggtttctaagcgtgaagagtatgagaaagacttcggtcgcgacttcaccgaacgtaaatgttcccaaatcatttctcgtgccagcatgctgatggttgcagtggtgatgttctttgcctttagctgcctgtttactctgtctccggccaacatggcggaagccaaagcgcagaatattccagtgctttcttatctggctaaccactttgcgtccatgaccggtaccaaaacaacgttcgcgattacactggaatatgcggcttccatcatcgcactcgtggctatcttcaaatctttcttcggtcactatctgggaacgctggaaggtctgaatggcctggtcctgaagtttggttataaaggcgacaaaactaaagtgtcgctgggtaaactgaacactatcagcatgatcttcatcatgggctccacctgggttgttgcctacgccaacccgaacatccttgacctgattgaagccatgggcgcaccgattatcgcatccctgctgtgcctgttgccgatgtatgccatccgtaaagcgccgtctctggcgaaataccgtggtcgtctggataacgtgtttgttaccgtgattggtctgctgaccatcct

SEQ ID NO: 34 - DNA, 1254 bp, Artificial Sequence
  Synthetic DNA
atgacattctccctttttggtgacaaatttacccgccactccggcattacgctgttgatggaagatctgaacgacggtttacgcacgcctggcgcgattatgctcggcggcggtaatccggcgcagatcccggaaatgcaggactacttccagacgctactgaccgacatgctggaaagtggcaaagcgactgatgcactgtgtaactacgacggtccacaggggaaaacggagctactcacactgcttgccggaatgctgcgcgagaagttgggttgggatatcgaaccacagaatattgcactaacaaacggcagccagagcgcgtttttctacttatttaacctgtttgccggacgccgtgccgatggtcgggtcaaaaaagtgctgttcccgcttgcaccggaatacattggctatgctgacgccggactggaagaagatctgtttgtctctgcgcgtccgaatattgaactgctgccggaaggccagtttaaataccacgtcgattttgagcatctgcatattggcgaagaaaccgggatgatttgcgtctcccggccgacgaatccaacaggcaatgtgattactgacgaagagttgctgaagcttgacgcgctggcgaatcaacacggcattccgctggtgattgataacgcttatggcgtcccgttcccgggtatcatcttcagtgaagcgcgcccgctatggaatccgaatatcgtgctgtgcatgagtctttccaagctgggtctacctggctcccgctgcggcattatcatcgccaatgaaaaaatcatcaccgccatcaccaatatgaacggcattatcagcctggcacctggcggtattggtccggcgatgatgtgtgaaatgattaagcgtaacgatctgctgcgcctgtctgaaacagtcatcaaaccgttttactaccagcgtgttcaggaaactatcgccatcattcgccgctatttaccggaaaatcgctgcctgattcataaaccggaaggagccattttcctctggctatggtttaaggatttgcccattacgaccaagcagctctatcagcgcctgaaagcacgcggcgtgctgatggtgccggggcacaacttcttcccagggctggataaaccgtggccgcatacgcatcaatgtatgcgcatgaactacgtaccagagccggagaaaattgaggcgggggtgaagattctggcggaagagatagaaagagcctgggctgaaagtcactaa

SEQ ID NO: 35 - DNA, 1218 bp, Artificial Sequence
  Synthetic DNA
atgtcccccattgaaaaatccagcaaattagagaatgtctgttatgacatccgtggtccggtgctgaaagaagcaaaacgcctggaagaagaaggtaacaaggtactgaaactgaacatcggcaacccagccccgttcggttttgacgcgccagatgaaatcctcgttgacgtgatacgcaacctgcctaccgctcaagggtattgcgattccaaaggtctttactccgcgcgtaaagccatcatgcagcactaccaggctcgtggcatgcgtgatgttaccgtggaagatatttacatcggcaatggtgtatcggagcttatcgttcaggcaatgcaggcattgctgaacagcggggacgaaatgttggttcctgcaccagattacccactctggaccgcggcggtttcgctttccagcggtaaagcggtgcattatctttgcgatgaatcctctgactggttcccggacctcgatgatattcgcgctaaaattacgcctcgtacgcgtgggatcgttattatcaacccaaataacccaaccggcgcggtatattccaaagagcttttaatggagattgtggagattgcacgtcagcataatctcattatcttcgccgatgaaatttatgacaaaattctctacgacgacgctgagcatcactcaattgcgccgctggcacctgacctgctgaccattacctttaacggactgtcgaaaacgtaccgcgttgcaggcttccgtcaggggtggatggtgttgaacgggccgaaaaaacacgccaaaggctacatcgaaggtctggaaatgctggcttcaatgcgcctgtgtgctaacgttcctgcgcaacacgccattcagaccgcgctaggtggttatcagagcatcagtgaatttattacccctggcggtcgtctttatgagcagcgtaaccgcgcgtgggaactgatcaacgatattccgggcgtttcctgcgtgaaacctcgtggtgcgctgtatatgttcccgaaaatcgacgccaaacgctttaacattcacgacgatcagaaaatggtgttggatttcctgttgcaggaaaaagttctgttggtgcaagggacggcattcaactggccgtggccggatcacttccgcattgtcacgctaccgcgtgtcgatgatatcgagctgtctttgagcaagttcgcgcgtttcctttctggttatcatcagctgtaa

SEQ ID NO: 36 - DNA, 1239 bp, Artificial Sequence
  Synthetic DNA
atggctgacactcgccctgaacgtcgctttacgcgcattgatcgtctcccgccctacgtttttaacatcaccgctgaactgaaaatggctgcgcgtcggcgcggcgaagatattatcgatttcagcatgggtaacccggacggtgcgactccgccgcatatcgtcgaaaaattatgtactgtggcccagcgcccggacacgcatggttactccacttcacgcggcattccgcggttacgtcgcgccatttcccgctggtatcaggatcgctacgacgttgaaatcgacccggaatcagaagccatcgtcactattggttcgaaagagggcctggcgcatctgatgctggcgacgctggatcatggtgacacggtgctggtgccgaatccaagttacccgatccatatttatggcgcggtgattgccggggcgcaggtacgctcagtgccgctggtggaaggtgtcgatttcttcaacgaactggagcgagccattcgtgaaagttatccgaaaccgaagatgatgatcctcggcttcccgtcaaacccaaccgcgcaatgcgtggaactggagttcttcgaaaaagtagtggcgctggcgaaacgctacgatgtgctggtggtccatgacctggcctatgccgatatcgtctacgatggctggaaagcgccgtcaattatgcaggtaccgggtgcacgcgatgtggcagtcgagttctttacgctgtcgaaaagctacaacatggcgggctggcgtatcggctttatggttggcaacaaaacgctggtcagcgccctggctcgtattaaaagctatcacgattacggcacctttacgccgttgcaggtggcggcgattgcggcgctggagggcgatcaacagtgcgtgcgcgacattgccgaacagtacaaacgccgccgtgatgtattggttaaagggctgcatgaagcgggctggatggttgaaatgccgaaggcttcgatgtatgtctgggcgaaaatcccggaaccatatgcggccatgggatcgctggaatttgccaagaagctccttaacgaagcgaaggtttgtgtctcgccggggattggctttggcgactatggtgatacgcatgttcgctttgcactgattgaaaaccgcgaccgtattcgtcaggccatcagagggattaaagcgatgttccgtgccgacggtttactacccgccagcagcaaacatattcacgaaaacgcggaataa

SEQ ID NO: 37 - DNA, 59 bp, Artificial Sequence
  Synthetic DNA
tggagctctctagaactagataagaggtatatattaatgttgatgttatttctcaccgt

SEQ ID NO: 38 - DNA, 42 bp, Artificial Sequence
  Synthetic DNA
cttcctgcaggatccactagtcgccttatccgacttactctg

SEQ ID NO: 39 - DNA, 57 bp, Artificial Sequence
  Synthetic DNA
tggagctctctagaactagaaggagatatacatatgaaaaaactactcgtcgccaat

SEQ ID NO: 40 - DNA, 57 bp, Artificial Sequence
  Synthetic DNA
tggagctctctagaactagaaggagatatacatatgaaaaaactgctggtcgcaaat

SEQ ID NO: 41 - DNA, 58 bp, Artificial Sequence
  Synthetic DNA
aagtcggataaggcgactagaaggagatatacatatgaaaaaactgctggtcgcaaat

SEQ ID NO: 42 - DNA, 55 bp, Artificial Sequence
  Synthetic DNA
cttcctgcaggatccactagttacgtcaaagttagtcaatttcaatcaataagtc

SEQ ID NO: 43 - DNA, 40 bp, Artificial Sequence
  Synthetic DNA
tgaccgaatacattggaattgtgctgttttgtaacccgcc

SEQ ID NO: 44 - DNA, 63 bp, Artificial Sequence
  Synthetic DNA
tgaccgaatacattggaattacacaggaaacctactaggtgtcccgtattattatgctgatcc

SEQ ID NO: 45 - DNA, 41 bp, Artificial Sequence
  Synthetic DNA
acctcgagtgatatcgaattcactgcggatgatgacgagat

SEQ ID NO: 46 - DNA, 85 bp, Artificial Sequence
  Synthetic DNA
acctcgagtgatatcgaattcattaagctgctaaagcgtagttttcgtcgtttgctgcctgctgctgtgcagactgaatcgcagt

SEQ ID NO: 47 - DNA, 65 bp, Artificial Sequence
  Synthetic DNA
tgaccgaatacattggaattaaagaggagaaaggattcatggctgatacaaaagcaaaactcacc

SEQ ID NO: 48 - DNA, 39 bp, Artificial Sequence
  Synthetic DNA
tgaccgaatacattggaattacgcaataaggcgctaagg

SEQ ID NO: 49 - DNA, 84 bp, Artificial Sequence
  Synthetic DNA
acctcgagtgatatcgaattcattaagctgctaaagcgtagttttcgtcgtttgctgcacgcttgatatcgcttttaaagtcgc

SEQ ID NO: 50 - DNA, 46 bp, Artificial Sequence
  Synthetic DNA
tgaccgaatacattggaattggagtacattgttctaagctgacttc

SEQ ID NO: 51 - DNA, 61 bp, Artificial Sequence
  Synthetic DNA
tgaccgaatacattggaattacacaggaaacctactagatgtcccccattgaaaaatccag

SEQ ID NO: 52 - DNA, 80 bp, Artificial Sequence
  Synthetic DNA
acctcgagtgatatcgaattcattaagctgctaaagcgtagttttcgtcgtttgctgccagctgatgataaccagaaagg

SEQ ID NO: 53 - DNA, 46 bp, Artificial Sequence
  Synthetic DNA
tgaccgaatacattggaattggacagacagaaattaatcaggctat

SEQ ID NO: 54 - DNA, 61 bp, Artificial Sequence
  Synthetic DNA
tgaccgaatacattggaattacacaggaaacctactagatggctgacactcgccctgaacg

SEQ ID NO: 55 - DNA, 80 bp, Artificial Sequence
  Synthetic DNA
acctcgagtgatatcgaattcattaagctgctaaagcgtagttttcgtcgtttgctgcttccgcgttttcgtgaatatgt

SEQ ID NO: 56 - DNA, 546 bp, Artificial Sequence
  Synthetic DNA
tttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaa

SEQ ID NO: 57 - DNA, 693 bp, Artificial Sequence
  Synthetic DNA
atgtctaaaggtgaagaattattcactggtgttgtcccaattttggttgaattagatggtgatgttaatggtcacaaattttctgtccgcggtgaaggtgaaggtgatgctactatcggtaaattgaccttaaaatttatttgtactactggtaaattgccggttccatggccaaccttagtcactactttcggttatggtgttcaatgttttgctagatacccagatcacatgaaacaacatgactttttcaagtctgccatgccagaaggttatgttcaagaaagaactatttttttcaaagatgacggtaaatacaagaccagagctgtagtcaagtttgaaggtgataccttagttaatagaatcgaattaaaaggtactgattttaaagaagatggtaacattttaggtcacaaattggaatacaactttaactctcacaatgtttacatcatggctgacaaacaaaagaatggtatcaaagttaacttcacagtaagacacaacgttgaagatggttctgttcaattagctgaccattatcaacaaaatactccaattggtgatggtccagtcttgttaccagacaaccattacttatccactcaaactgtcttatccaaagatccaaacgaaaagagagaccacatggtcttgcatgaatatgttaatgctgctggtattacctaa

(19) United States
(12) Patent Application Publication
(10) Pub. No.: US 2024/0060076 A1
(43) Pub. Date: Feb. 22, 2024

(54) THERMAL SWITCH SYSTEM AND APPLICATION THEREOF IN IMPROVING YIELD OF AMINO ACID

(71) Applicant: Jiangnan University, Wuxi (CN)

(72) Inventors: Xiaoyuan WANG, Wuxi (CN); Yu FANG, Wuxi (CN); Jianli WANG, Wuxi (CN); Shuyan ZHANG, Wuxi (CN); Xiaoqing HU, Wuxi (CN)

(73) Assignee: Jiangnan University, Wuxi (CN)

(21) Appl. No.: 17/746,130

(22) Filed: May 17, 2022

Related U.S. Application Data
(63) Continuation of application No. PCT/CN2020/138263, filed on Dec. 22, 2020.

(30) Foreign Application Priority Data
Jan. 19, 2020 (CN) 2020100571446

(51) Int. Cl. C12N 15/70; C12P 13/08

(52) U.S. Cl. CPC C12N 15/70; C12P 13/08

(57) ABSTRACT

The disclosure relates to a thermal switch system and application thereof in improving yield of amino acid, and particularly relates to a method for improving the yield of amino acid by regulating intracellular metabolic flux distribution using the thermal switch system, which belongs to the technical fields of genetic engineering and microbial fermentation. The system rebalances metabolic flux between pyruvate and oxaloacetate by controlling heterologous expression of pyruvate carboxylase and in combination with chemical properties that oxaloacetate is temperature-sensitive and easy to decarboxylate, and dynamically regulates a central metabolic pathway to ensure the supply of reducing cofactors, so as to promote the production of L-threonine. Temperature-controlled threonine-producing strains TWF106/pFT24rp and TWF113/pFT24rpa1 obtained in the disclosure have threonine molar conversion rates of 111.78% and 124.03% respectively.

### INCORPORATION BY REFERENCE STATEMENT REGARDING THE MATERIAL ELECTRONICALLY SUBMITTED

Incorporated by reference in its entirety herein is a computer-readable nucleotide/amino acid sequence listing submitted and identified as: “sequence listing.txt” with “86,779 bytes” and created on: Jan. 30, 2023.

### CROSS-REFERENCE TO RELATED APPLICATIONS

The application claims priority to Chinese patent application No. 202010057144.6 filed on Jan. 19, 2020, the entire contents of which are incorporated herein by reference.

### TECHNICAL FIELD

The disclosure relates to a thermal switch system and application thereof in improving yield of amino acid, and particularly relates to a method for improving the yield of amino acid by regulating intracellular metabolic flux distribution using the thermal switch system, which belongs to the technical fields of genetic engineering and microbial fermentation.

### BACKGROUND

The combination of metabolic engineering and metabolic regulation has been adopted to improve the yield of natural chemicals, especially large-volume amino acid products. However, unbalanced cellular metabolic flux distribution between cell growth and target products has limited the yield of products and further improvement of productivity for a long time. Traditional techniques(such as inactivating related genes in alternative pathways and overexpressing related genes in heterologous pathways) cannot handle with challenges of more complicated carbon distribution (such as requiring a plurality of cofactors to participate in synthesis of the products).

Excessive heterologous pathways may compete with cellular energy-supplying metabolic pathways, and transform more carbon sources into intermediate metabolites, which result in growth arrest and reduction in production yield and productivity. In recent years, the method of optimizing the metabolic flux distribution by regulating the gene expression level has made certain progress, such as constructing engineering promoters, regulating the strength of ribosome binding sequences, expressing small regulatory RNA and the like. Although these technologies can effectively and rapidly regulate the gene expression level to improve the production performance of engineering strains, these regulating manners are static control manners, and are difficult to respond to dynamic metabolic environments, resulting in that a target product is synthesized under a suboptimum state by the strains.

At present, the yield and productivity of the engineering strains may be effectively improved by dynamically regulating the metabolic pathways through a synthetic biosensor and by redirecting the metabolic flux through a gene circuit. After being synthesized, these biosensors are designed to produce fatty acid ethyl ester, glucaric acid, 2-fucosyllactose, γ-aminobutyric acid and other substances. However, most of the biosensors require huge workload of testing by gene circuit elements, so as to adapt to a target strain under specific fermentation conditions. In addition, because there is a high risk of bacterial infection and high downstream purification cost in the process of tank fermentation, inducers are rarely used in industrial fermentation production.

The activity of a thermosensitive biosensor is controlled by temperature, which can reduce the metabolic burden of a host in the cell growth stage, and the thermosensitive biosensor has been applied to expression of recombinant protein, synthesis of D-lactic acid and synthesis of itaconic acid. Although the temperature-dependent regulating module has great industrial application prospect, due to its uncertainty, few related products are reported. For example, when the temperature regulating the thermosensitive biosensor changes, it may cause unknown influence on overall cellular metabolism and leakage expression of thermosensitive promoters.

Threonine, as a typical oxaloacetate derivative and essential amino acid which cannot be synthesized by humans and animals, has been capable of being industrially produced by microbial fermentation. So far, the threonine yield of engineering microbiology has been greatly improved, but the complicated metabolic flux distribution between the synthetic pathway and the TCA cycle has become a bottleneck for further improvement of threonine conversion rate. The highest molar conversion rate currently reported is only 87.8%. The threonine synthetic pathway competes with the central metabolic pathway for common precursor metabolites, but also requires the TCA cycle to provide multiple cofactors (NADPH and ATP), since the TCA cycle is the main energy-supplying pathway for cells to grow under aerobic conditions.

To improve the productivity of threonine, the precursor oxaloacetate, the main metabolic branch among glycolysis, L-aspartic acid and the TCA cycle, need to be continously supplied for the engineering strains. In the conventional methods, accumulation of oxaloacetate in Escherichia coli is mainly realized by overexpressing phosphoenolpyruvate carboxylase (PPC) or heterologously expressing phosphoenolpyruvate carboxykinase (PCK), and then catalyzing phosphoenolpyruvate, so as to produce oxaloacetate. However, relatively abundant pyruvate is generally decarboxylated and completely oxidized to carbon dioxide in the TCA cycle, resulting in accumulation of a large number of reducing factors (NADH, NADPH and ATP). Intracellular pyruvate synthetic pathways mainly include a pyruvate kinase and PTS system coupled pathway and an ED pathway, in which abundant pyruvate is easily wasted, and redundant energy substances and undesired by-products (acetate, formate, lactate and ethyl alcohol) are produced. These organic acid by-products may cause disruptions in glycolytic pathways and central carbon metabolism, thereby hindering cell growth and resulting in adverse product synthesis. Pyruvate carboxylase (PYC) is another heterologous pathway from pyruvate to oxaloacetate, and has been applied to produce multiple derivatives of TCA cycle intermediate metabolites in E. coli. In the previous studies, in case of without considering the supply of cofactors, the high-level expression of PYC makes as many as possible of carbon sources to be used to synthesize the target product. However, the low-level expression of PYC is hard to accumulate more oxaloacetate, which results in a relatively low conversion rate of oxaloacetate derivatives.

### SUMMARY

In order to solve the above problems, the disclosure constructs a method for dynamically regulating the metabolic flux of a central metabolic pathway, and the method improves the downstream product conversion rate of oxaloacetate and makes it has industrial applicability by rebalancing carbon distribution in microbial cells.

A first objective of the disclosure is to provide a thermal switch vector, which is obtained by connecting a thermal switch circuit onto a vector. The thermal switch circuit includes a temperature-sensitive circuit cIts-pR-pL and a rigorous circuit tetR-PLtetO-1. The temperature-sensitive circuit cIts-pR-pL is composed of a temperature-sensitive repressor gene cIts and a tandem promoter pR-pL, and a nucleotide sequence is shown as GenBank: AB248919.1. The rigorous circuit tetR-PLtetO-1 is composed of a repressor gene tetR and a promoter PLtetO-1, and a nucleotide sequence of the repressor gene tetR is shown as SEQ ID NO: 1. pMB1 in a plasmid pFW001 is replaced with a replicon p15A with a medium copy number, and a PJ23101 promoter in the plasmid pFW001 is replaced with the thermal switch circuit. The thermal switch circuit is configured to successively connect the temperature-sensitive repressor gene cIts, the promoter pR-pL, RBS, the repressor gene tetR, a multiple cloning site sequence MCS1, a terminator T7, the promoter PLtetO-1, a multiple cloning site sequence MCS2 and a terminator T1 in series. The RBS is high strength RBS or low strength RBS; a nucleotide sequence of the high strength RBS is shown as SEQ ID NO: 7; and a nucleotide sequence of the low strength RBS is shown as SEQ ID NO: 8.

In one embodiment, when the RBS is the low strength RBS, a vector pFT24 is obtained.

In one embodiment, when the RBS is the high strength RBS, a vector pFT22 is obtained.

In one embodiment, when the RBS is the low strength RBS, genes rhtC and pycmt are successively inserted at the MCS1 of the vector, and the genes rhtC and pycmt are co-expressed under the control of a cIts-pR-pL circuit to obtain a thermal switch vector pFT24rp. Alternatively, when the RBS is the low strength RBS, a gene rhtC or a gene pyc is inserted at the MCS1 of the vector to obtain pFT24r and pFT24p respectively.

In one embodiment, on the basis of the thermal switch vector pFT24rp, a gene alaA labeled with a standard SsrA degraded peptide chain is inserted in the MCS2 to obtain a thermal switch vector pFT24rpa1; and an amino acid sequence of the standard SsrA degraded peptide chain is shown as SEQ ID NO: 10.

In one embodiment, the thermal switch vector is to replace the pMB1 in the plasmid pFW001 with the replicon p15A with a medium copy number, and replace the PJ23101 promoter in the plasmid pFW001 with the thermal switch circuit. The thermal switch circuit is configured to successively connect the temperature-sensitive repressor gene cIts, the promoter pR-pL, the RBS, the repressor gene tetR, the multiple cloning site sequence MCS1, the terminator T7, the promoter PLtetO-1, the multiple cloning site sequence MCS2 and the terminator T1 in series.

In one embodiment, when the nucleotide sequence of the RBS is shown as SEQ ID NO: 7, the pFT22 is obtained. The thermal switch vector includes the pFT22 or a recombinant vector constructed on the basis of the pFT22.

In one embodiment, when the nucleotide sequence of the RBS is shown as SEQ ID NO: 8, the pFT24 is obtained. The thermal switch vector includes the pFT24 or a recombinant vector constructed on the basis of the pFT24.

In one embodiment, the thermal switch vector includes pFT24r, pFT24p, pFT24pm, pFT24rp, pFT24t1, pFT24t2, pFT24t3, pFT24t4, pFT24rpt3, pFT24rpa1, pFT24rpa2, pFT24rpa3 or pFT24rpa4.

In one embodiment, the nucleotide sequence of the RBS of the vector pFT24r, pFT24p, pFT24pm, pFT24rp, pFT24t1, pFT24t2, pFT24t3, pFT24t4, pFT24rpt3, pFT24rpa1, pFT24rpa2, pFT24rpa3 or pFT24rpa4 is shown as SEQ ID NO: 8.

In one embodiment, the vector pFT24r is obtained by inserting a gene rhtC at the MCS1 of the vector pFT24.

In one embodiment, the vector pFT24p is obtained by inserting a gene pyc at the MCS1 of the vector pFT24.

In one embodiment, the vector pFT24pm is obtained by inserting a gene pycmt at the MCS1 of the vector pFT24.

In one embodiment, the vector pFT24rp is obtained by successively inserting genes rhtC and pycmt at the MCS1 of the vector pFT24.

In one embodiment, the vector pFT24t1 is obtained by inserting a gene pta at the MCS2 of the vector pFT24.

In one embodiment, the vector pFT24t2 is obtained by inserting a gene pta connected with an RBS sequence with the nucleotide sequence being shown as SEQ ID NO: 9 at the MCS2 of the vector pFT24.

In one embodiment, the vector pFT24t3 is obtained by inserting a gene pta labeled with a standard SsrA degraded peptide chain at the MCS2 of the vector pFT24.

In one embodiment, the vector pFT24t4 is obtained by inserting a gene pta labeled with the standard SsrA degraded peptide chain and connected with the RBS sequence with the nucleotide sequence being shown as SEQ ID NO: 9 at the MCS2 of the vector pFT24.

In one embodiment, the vector pFT24rpt3 is obtained by successively inserting genes rhtC and pycmt at the MCS1 of the vector pFT24 and inserting a gene pta labeled with the standard SsrA degraded peptide chain and containing an original RBS on the genome in the MCS2.

In one embodiment, the vector pFT24rpa1 is obtained by successively inserting the genes rhtC and pycmt at the MCS1 of the vector pFT24 and inserting a gene alaA labeled with the standard SsrA degraded peptide chain and containing the original RBS on the genome in the MCS2.

In one embodiment, the vector pFT24rpa2 is obtained by successively inserting the genes rhtC and pycmt at the MCS1 of the vector pFT24 and inserting a gene alaA labeled with the standard SsrA degraded peptide chain and connected with the RBS sequence with the nucleotide sequence being shown as SEQ ID NO: 9 in the MCS2.

The vector pFT24rpa3 is obtained by successively inserting the genes rhtC and pycmt at the MCS1 of the vector pFT24 and inserting a gene alaC labeled with the standard SsrA degraded peptide chain in the MCS2.

The vector pFT24rpa4 is obtained by successively inserting the genes rhtC and pycmt at the MCS1 of the vector pFT24 and inserting a gene alaC labeled with the standard SsrA degraded peptide chain and connected with the RBS sequence with the nucleotide sequence being shown as SEQ ID NO: 9 in the MCS2.

The amino acid sequence of the standard SsrA degraded peptide chain is shown as SEQ ID NO: 10, and the nucleotide sequence of the standard SsrA degraded peptide chain is shown as SEQ ID NO: 11.

In one embodiment, a temperature control range of the thermal switch vector is between 30° C. and 42° C.

A second objective of the disclosure is to provide a method for regulating a relative level between pyruvate and oxaloacetate in cells. The method uses the above thermal switch vector to control the expression of pyruvate carboxylase, and stops the expression of pyruvate carboxylase at the cell growth stage of fermentation, so as to ensure rapid accumulation of the strain biomass. When sufficient fermentation biomass is accumulated, the expression of pyruvate carboxylase is started to provide sufficient oxaloacetate for a target product to be synthesized.

In one embodiment, the method combines with the chemical properties of oxaloacetate, and the higher temperature can accelerate the spontaneous decarboxylation of oxaloacetate (see FIG. 7). When a strain is synthesized into threonine from glucose, more oxaloacetate is required to provide precursor intermediate metabolites, and at the same time, sufficient pyruvic acid is required to be oxidized to generate enough reducing power (NADPH, NADH and ATP). The metabolic flux distribution of the central metabolic pathway is dynamically regulated by controlling the expression of pyruvate carboxylase through the thermal switch vector in combination with the chemical property of oxaloacetate (see FIG. 1).

A third objective of the disclosure is to provide a threonine-producing strain, which expresses the above thermal switch vector.

In one embodiment, the thermal switch vector includes pFT24, pFT22, pFT24r, pFT24p, pFT24pm, pFT24rp, pFT24t1, pFT24t2, pFT24t3, pFT24t4, pFT24rpt3, pFT24rpa1, pFT24rpa2, pFT24rpa3 or pFT24rpa4.

Preferably, the strain expresses the thermal switch vector pFT24rp, pFT24rpa1 or pFT24.

In one embodiment, the thermal switch vector is transferred to a threonine-producing platform strain, and the threonine-producing platform strain includes E. coli TWF001, TWF101, TWF102, TWF103, TWF104, TWF105, TWF106, TWF107, TWF108, TWF110, TWF111, TWF112 or TWF113. The TWF001 overexpresses a gene pntAB encoding pyridine nucleotide transhydrogenase and related genes ppc, aspC, lysC, asd, thrAG433RBC and rhtA involving in threonine production, and the construction method is disclosed in the following document: Zhao, H., Fang, Y., Wang, X., Zhao, L., Wang, J., Li, Y., 2018. Increasing L-threonine production in Escherichia coli by engineering the glyoxylate shunt and the L-threonine biosynthesis pathway. The TWF101, TWF102, TWF103, TWF104, TWF105, TWF106, TWF107, TWF108, TWF110, TWF111, TWF112 and TWF113 are obtained as follows: on the basis of the TWF001, unimportant genes poxB, pflB, ldhA, adhE, pta related to organic acid synthesis, a gene tdcC encoding threonine translocator, and genes avtA, alaA, alaC related to alanine synthetic pathways are further knocked out independently or in combination (see FIG. 2), and the specific construction method is shown in Table 1.

In one embodiment, the E. coli TWF106 is obtained by knocking out poxB, pflB, ldhA, adhE and tdcC in the TWF001.

In one embodiment, the E. coli TWF113 is obtained by knocking out poxB, pflB, ldhA, adhE, tdcC, avtA, alaA and alaC in the TWF001.

In one embodiment, on the basis of the TWF106, the strain uses the thermal switch vector to control the independent expression or combined expression of the gene rhtC encoding threonine extracellular translocator, the gene pyc encoding pyruvate carboxylase and the gene pycmt encoding pyruvate carboxylase based on pyc codon optimization, so as to obtain strains TWF106/pFT24r, TWF106/pFT24p, TWF106/pFT24p and TWF106/pFT24rp with high threonine yield, of which the threonine yields are 17.24 g/L, 20.15 g/L, 20.60 g/L and 23.29 g/L respectively, and the corresponding molar glucose-acid conversion rates are 72.44%, 81.50%, 98.43% and 111.78% respectively.

In one embodiment, on the basis of the TWF106/pFT24rp, the strain further uses the thermal switch vector to close the alanine synthetic pathway to obtain a strain TWF113/pFT24rpa1 with higher threonine conversion rate.

In one embodiment, the construction method of the TWF113/pFT24rpa1 is as follows: on the basis of the TWF106, three genes avtA, alaA and alaC are simultaneously knocked out to obtain the TWF113; on the basis of the thermal switch vector pFT24rpa1, the promoter PLtetO-1 is used to control the expression of alaA to obtain the expression vector pFT24rpa1; the pFT24rpa1 is transferred into TWF113, so as to obtain the TWF113/pFT24rpa1, of which the threonine yield is 25.85 g/L, and the molar glucose-acid conversion rate is 124.03%.

A fourth objective of the disclosure is to provide a method for producing threonine, which takes the above threonine-producing strain as a fermentation strain, and produces threonine by fermentation.

In one embodiment, the strain includes TWF106/pFT24r, TWF106/pFT24p, TWF106/pFT24p, TWF106/pFT24rp and TWF113/pFT24rpa1.

In one embodiment, a fermentation strain seed culture with an initial OD600 of 0.2 to 0.3 is inoculated into a fermentation medium, and fermentation culture is performed until glucose is completely consumed.

In one embodiment, a fermentation strain seed culture with the initial OD600 of 0.2 to 0.3 is inoculated into a fermentation medium; fermentation culture is performed at 36° C. to 38° C. for 5 h to 8 h; and culture is continued at 41° C. to 43° C. until glucose in a fermentation broth is completely consumed.

In one embodiment, a fermentation strain seed culture is obtained by culturing the strain in an STF seed medium.

In one embodiment, the seed medium is an STF medium, with the formula as follows: 10 g/L saccharose, 20 g/L peptone, 5 g/L yeast extract, 15 g/L (NH4)2SO4, 1 g/L MgSO4, and pH 7.3. The formula of the fermentation medium is as follows: 35 g/L glucose, 25 g/L (NH4)2SO4, 7.46 g/L KH2PO4, 2 g/L yeast extract, 2 g/L citric acid, 2 g/L MgSO4·7 H2O, 5 mg/L FeSO4·7 H2O, 5 mg/L MnSO4.4 H2O and pH 7.1.

A fifth objective of the disclosure is to provide application of the above thermal switch vector in producing protein.

A sixth objective of the disclosure is to provide application of the above thermal switch vector in producing amino acids of the aspartic acid family and derivatives thereof.

In one embodiment of the disclosure, the amino acids of the aspartic acid family and derivatives thereof include aspartic acid, homoserine, threonine, lysine, methionine, isoleucine and derivatives thereof.

Advantages and effects of the disclosure:

(1) The disclosure constructs a thermal switch system to regulate the intracellular metabolic flux distribution to promote the production of threonine. The switch system includes a thermal switch vector, which has a replicon p 15A with a medium copy number, a triclosan-resistant screening gene fabV and two metabolic pathway-independent regulating circuit modules (a temperature-sensitive circuit cIts-pR-pL and a rigorous circuit tetR-PLtetO-1). After being gradually optimized and upgraded, the system is finally used to improve the yield of threonine. After being upgraded and improved, the switch temperature control range of the thermal switch vector is between 30° C. and 42° C., and the effective narrowest temperature control range is between 37° C. and 40° C.

(2) According to the disclosure, the thermal switch system is a metabolic pathway-independent regulating system, is not affected by an intracellular metabolic environment, and can optionally control the overexpression of target genes and stop the expression of other genes in the metabolic pathways. By weakening the RBS strength of a repressor TetR, the expression leakage of the promoter pR-pL is effectively relieved, the expression inhibition of the rigorous circuit tetR-PLtetO-1 under ambient temperature conditions is weakened, and the target genes are enabled to have a sufficient expression level before the switch is turned on.

(3) The thermal switch system of the disclosure is applied to an engineering strain for staged fermentation. The whole fermentation process can be divided into two stages of cell growth and fermentation production, thus the engineering strain has dual advantages of rapid biomass accumulation and efficient fermentation production at the same time, so as to further improve the production performance of the strain and shorten the fermentation cycle.

(4) The thermal switch system of the disclosure is applied to fermentation production, where inducers and antibiotics are not needed to be added, and only a small amount of trichlorine is added to maintain the plasmids to be stable. Furthermore, the switch system is very suitable for being combined with factory-scale fermentation equipment to realize industrial mass production.

(5) The thermal switch system of the disclosure is used to overexpress pyruvate carboxylase, combined with the chemical property that oxaloacetate is decarboxylated under heat, a ratio of pyruvate to oxaloacetate in the central metabolic pathway can be intelligently regulated, so as to achieve the purpose of greatly improving the yield of downstream products of oxaloacetate and simultaneously ensure the supply of sufficient cofactors (NADPH and ATP).

(6) The thermal switch system of the disclosure is applied to the threonine-producing platform strain, which greatly increases the threonine conversion rate, and two threonine-producing strains TWF106/pFT24rp and TWF113/pFT24rpa 1 are obtained. After temperature-variable shake-flask fermentation, the threonine yields are 23.29 g/L and 25.85 g/L respectively, and the corresponding threonine molar conversion rates are 111.78% and 124.03% respectively.

### BRIEF DESCRIPTION OF DRAWINGS

FIG. 1: A schematic diagram showing that a thermal switch system dynamically regulates the intracellular oxaloacetate level. Bold lines represent that genes in the metabolic pathways are overexpressed.

FIG. 2: Gene knockout of alternative pathways involved in platform strain construction.

FIG. 3: A schematic diagram showing constituting elements of a thermal switch vector and circuit regulation.

FIG. 4: A schematic diagram showing transformation and optimization of a thermal switch vector.

FIG. 5: Feasibility detection of a thermal switch system to control gene expression from aspects of translation level and transcription level.

FIG. 6: Sensitivity detection of a thermal switch system to control gene expression from aspects of translational level and transcriptional level.

FIG. 7: Influences of temperature and time on spontaneous decarboxylation of oxaloacetate.

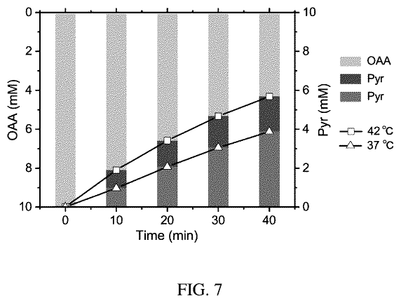

FIG. 8: Optimization of a seed medium for shake-flask fermentation and a comparison diagram showing that a threonine-producing strain TWF001 takes LB and STF as the seed medium to perform threonine fermentation.

FIG. 9: Optimization of a seed medium for shake-flask fermentation and a comparison diagram showing that a threonine-producing strain TWF001 takes LB and STF as the seed medium to perform threonine fermentation.

FIG. 10: A comparison diagram showing threonine shake-flask fermentation of a chassis strain with alternative pathway genes being knocked out under constant temperature fermentation at 37° C. and temperature-variable fermentation conditions.

FIG. 11: Threonine production shake-flask fermentation results that a thermal switch system regulates threonine extracellular transport protein RhtC and pyruvate carboxylase (PYC) expression.

FIG. 12: A schematic diagram showing that a thermal switch system controls the gene pta to close.

FIG. 13: Threonine production shake-flask fermentation results of a threonine-producing strain that controls the gene pta to close.

FIG. 14: Threonine production shake-flask fermentation results of a threonine-producing strain that controls the gene pta to close.

FIG. 15: A schematic diagram showing that a thermal switch system controls an L-alaine synthetic pathway to close.

FIG. 16: Threonine production shake-flask fermentation results of a threonine-producing strain that controls an L-alaine synthetic pathway to close.

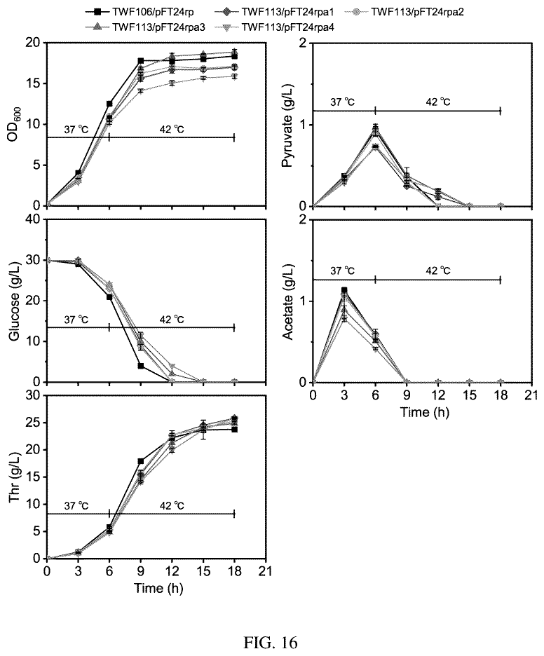

FIG. 17: A schematic diagram showing that a thermal switch system regulates the threonine-producing strain to gradually increase the threonine conversion rate.

### DETAILED DESCRIPTION OF THE EMBODIMENTS

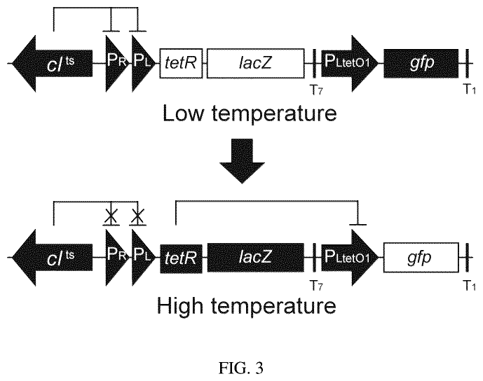

1. Working mechanism of thermal switch system: A thermosensitive CIts repressor is combined with a pR-pL promoter at room temperature and inhibits transcription, and a high temperature condition causes the CIts to be inactivated and the pR-pL promoter to be activated. A TetR repressor may inhibit transcription of a PLtetO-1 promoter, and a corresponding gene tetR is controlled by the pR-pL promoter. When detecting the effect of the thermal switch system, β-galactosidase LacZ and green fluorescent protein GFP are used as reporter proteins, and are respectively expressed under the pR-pL promoter and the PLtetO-1 promoter. An SsrA degraded tail is added to the C-terminal of GFP to eliminate GFP residual protein, so as to achieve the effect of complete close of genes. Under a low temperature condition, the thermosensitive CIts repressor is combined with the pR-pL promoter and inhibits transcription of downstream genes tetR and lacZ, then the PLtetO-1 promoter and gfp may normally transcribe. Under a high temperature condition, the CIts repressor protein is inactivated, and cannot be combined with the pR-pL promoter, so that the downstream genes tetR and lacZ normally transcribe, and the transcription of the PLtetO-1 promoter and gfp are inhibited (see FIG. 3).

When the thermal switch system is applied to a threonine-producing strain, a thermal switch vector can control some genes necessary for cell growth or influencing the cell growth, and start expression and stop expression at different fermentation stages. Acetic acid synthetic pathways and alanine synthetic pathways are necessary for strain growth and biomass accumulation, but these two alternative pathways may compete with a target product synthetic pathway for carbon sources, which will reduce the conversion rate of the target product, so that they need to express at the cell growth stage and switched off at the fermentation stage. However, the overexpression of pyruvate carboxylase may compete for the carbon sources entering the TCA cycle, which will hinder the normal growth of the strain; on the other hand, the expression of pyruvate carboxylase may accumulate more precursor intermediate metabolite oxaloacetate, which may promote the production of threonine and increase the glucose-acid conversion rate of the target product, so that the expression thereof needs to be stopped at the cell growth stage and started at the fermentation stage. The gene expression control described above can be realized by the thermal switch vector through temperature change.

2. Gene Knockout Method

A CRISPR-Cas9 knockout system is used to effectively edit an E. coli genome. An editing plasmid pCas is electrically transferred into threonine-producing E. coli at first so as to obtain a strain containing pCas, and the plasmid includes a gene cas9 with constitutive expression and a gene encoding λ Red recombinase with arabinose-induced expression. The strain containing pCas is cultured overnight (8 h to 14 h) to enable the initial OD600 to be 0.04, inoculated into an LB medium and added with 50 mg/L kanamycin and 10 mM arabinose, and then grows at 200 rpm under 30° C. until the OD600 reaches 0.6. 50 mL of induction-cultured pCas-containing strain culture is collected, and washed with ice-bathed 10% glycerite three times, then 2 mL of ice-bathed 10% glycerite is added to suspend the strain, and the solution is subpackaged into 1.5 mL EP tubes and stored at −70° C., so as to obtain competent cells with pCas.

An original pTargetF vector (pMB1 replicon, spectinomycin-resistant) is taken as a template, and a primer embedded with a target gene N20 sequence is used to amplify the whole plasmid. The template plasmid is eliminated by digesting a PCR product with DpnI, is transformed into E. coli DH5a, and an annular plasmid is generated through a terminal overlapping sequence.

The construction of the gene knockout strain is carried out according to the report of an existing literature (Jiang Y, Chen B, Duan C, et al. Multigene Editing in the Escherichia coli Genome via the CRISPR-Cas9 System). For example, poxB is knocked out; the above PCR product is transformed into E. coli DH5a; a transformant is selected and cultured overnight; and the plasmid pTargetF-poxB (containing the N20 sequence targeting the poxB locus) is extracted. A knockout template fragment with two homologous arms corresponding to the upstream and downstream regions of the poxB locus is obtained by overlap extension PCR. The pTargetF-poxB and the knockout template fragment are jointly transformed into a strain TWF001 through electroporation, and a gene encoding the λ Red recombinase on the pCas plasmid in the strain has been induction-expressed; and a transformant is selected and cultured overnight. 1 mL of cell culture is concentrated by centrifuging, and is coated on an LB agar plate containing kanamycin (50 mg/L) and spectinomycin (50 mg/L) for screening. A verification primer is used to carry out colony PCR to obtain a gene poxB knockout strain; then 0.5 mM IPTG is added to remove pTargetF-poxB; and incubation is performed at 42° C. to remove the temperature-sensitive plasmid pCas. The construction of other knockout strains in the disclosure is the same as the above steps.

3. Determination of activity of β-galactosidase (LacZ) by colorimetric method: See the reference for details: Li, W., Zhao, X., Zou, S., Ma, Y., Zhang, K., Zhang, M. Scanning assay of beta-galactosidase activity.

4. Detection of relative transcription level of two reporter genes in strain by real-time quantitative PCR: See the reference for details: Livak, K. J., Schmittgen, T. D. Analysis of relative gene expression data using real-time quantitative PCR and the 2(-Delta Delta C(T)) Method.

5. Extracellular metabolite analysis method

The cell concentration of a fermentation sample at different time points is monitored in real time by using a UV-1800 spectrophotometer to measure the absorbancy of OD600. Centrifugation is performed at 13,000×g for 10 min, and the culture supernate is taken to analyze the extracellular metabolite content, mainly including residual glucose, amino acid and organic acid. The concentration of the residual glucose in the culture solution is measured by using a biosensor analyzer (SBA-40C, Biology Institute of Shandong Academy of Sciences). The amino acid is quantified on Agilent 1200 or 1260 series instruments through high performance liquid chromatography by using a Thermo ODS-2HYPERSIL C18 chromatographic column (250 mm×4.0 mm). The sample is derivatized using a commercial ortho-phthalaldehyde reagent solution (Agilent Technologies), with a sample size of 1μL. As for the amino acid analysis method process, see the following reference: Koros, A., Varga, Z., Molnar-Perl, I. Simultaneous analysis of amino acids and amines as their o-phthalaldehyde-ethanethiol-9-fluorenylmethyl chloroformate derivatives in cheese by high-performance liquid chromatography.

The threonine yield is determined by comparing with an amino acid standard substance (Sigma). The determination of the organic acid is performed using an Aminex HPX-87H chromatographic column (300 mm×7.8 mm; Bio-Rad Laboratories), with the chromatographic column temperature of 55° C. 5 mM sulfuric acid is used as a mobile phase, and the flow rate is 0.6 mL/min. Detection is performed at the emission wavelength of 210 nm using a DAD detector, with a sample size of 10 μL, and the UV spectrum of each component is scanned, including pyruvate, acetate, malic acid, fumaric acid radical, oxaloacetate and the like.

6. Medium and conditions for fermentation production of threonine

STF seed medium: 10 g/L saccharose, 20 g/L peptone, 5 g/L yeast extract, 15 g/L (NH4)2504, 1 g/L MgSO4, pH 7.3.

Fermentation medium: 35 g/L glucose, 25 g/L (NH4)2SO4, 7.46 g/L KH2PO4, 2 g/L yeast extract, 2 g/L citric acid, 2 g/L MgSO4·7 H2O, 5 mg/L FeSO4·7H2O, 5 mg/L MnSO4·4H2O, pH 7.1.

Shake-flask fermentation at constant temperature of 37° C.: A strain containing the thermal switch vector is inoculated into 50 mL of a sterile STF seed medium, and cultured under the conditions of 37° C. and 200 rpm for 5 h. The seed culture is inoculated into the sterile fermentation medium with the initial OD600=0.2, and fermentation culture is performed until glucose in a fermentation broth is completely consumed.

Temperature-variable shake-flask fermentation: A strain containing the thermal switch vector is inoculated into 50 mL of a sterile STF seed medium, and cultured under the conditions of 37° C. and 200 rpm for 5 h. The seed culture is inoculated into the sterile fermentation medium with the initial OD600=0.2, fermentation is performed for 6 h, then the temperature is raised to 42° C., and fermentation culture is performed until glucose in a fermentation broth is completely consumed.

The construction methods of the threonine-producing platform strains and the thermal switch vector involved in the disclosure are shown in Table 1, and the sources or sequences of genes or proteins are shown in Table 2.

TABLE 1
Construction methods of threonine-producing
platform strains and thermal switch vector

| Strain or plasmid | Construction method |
|---|---|
| Strain | |
| TWF001 | Zhao H, Fang Y, Wang X, et al. Increasing 1-threonine |
| | production in Escherichia coli by engineering the glyoxylate |
| | shunt and the 1-threonine biosynthesis pathway [J]. Applied |
| | Microbiology and Biotechnology, 2018. |
| TWF101 | TWF001 ΔlacZI |
| TWF102 | TWF001 ΔpoxB |
| TWF103 | TWF001 ΔpoxBΔpflB |
| TWF104 | TWF001 ΔpoxBΔpflBΔldhA |
| TWF105 | TWF001 ΔpoxBΔpflBΔldhAΔadhE |
| TWF106 | TWF001 ΔpoxBΔpflBΔldhAΔadhEΔtdcC |
| TWF107 | TWF001 ΔpoxBΔpflBΔldhAΔpta |
| TWF108 | TWF001 ΔpoxBΔpflBΔldhAΔadhEΔtdcCΔpta |
| TWF110 | TWF001 ΔpoxBΔpflBΔldhAΔadhEΔtdcCΔavtA |
| TWF111 | TWF001 ΔpoxBΔpflBΔldhAΔadhEΔtdcCΔavtAΔalaA |
| TWF112 | TWF001 ΔpoxBΔpflBΔldhAΔadhEΔtdcCΔavtAΔalaC |
| TWF113 | TWF001 ΔpoxBΔpflBΔldhAΔadhEΔtdcCΔavtAΔalaAΔalaC |
| Thermal switch | |
| vector | |
| pFT22 | Triclosan-resistant vector, p15A ori, λcI (ts), P_{RL}::tetR (high |
| | strength RBS), MCS1, P_{LtetO1}::MCS2 |
| pFT24 | Triclosan-resistant vector, p15A ori, λcI (ts), P_{RL}::tetR (low |
| | strength RBS), MCS1, P_{LtetO1}::MCS2 |
| pFT22-lacZ-gfp | P_{RL}::tetR (H), lacZ, P_{LtetO1}::gfp |
| pFT22-lacZ-gfp(LAA) | P_{RL}::tetR (H), lacZ, P_{LtetO1}::gfp(LAA) |
| pFT24-lacZ-gfp | P_{RL}::tetR, lacZ, P_{LtetO1}::gfp |
| pFT24-lacZ-gfp(LAA) | P_{RL}::tetR, lacZ, P_{LtetO1}::gfp(LAA) |
| pFT24r | P_{RL}::tetR, rhtC, P_{LtetO1}::MCS2 |
| pFT24p | P_{RL}::tetR, pyc, P_{LtetO1}::MCS2 |
| pFT24pm | P_{RL}::tetR, pycmt, P_{LtetO1}::MCS2 |
| pFT24rp | P_{RL}::tetR, rhtC, pycmt, P_{LtetO1}::MCS2 |
| pFT24t1 | P_{RL}::tetR, MCS1, P_{LtetO1}::pta (M) |
| pFT24t2 | P_{RL}::tetR, MCS1, P_{LtetO1}::pta (H) |
| pFT24t3 | P_{RL}::tetR, MCS1, P_{LtetO1}::pta(LAA) (M) |
| pFT24t4 | P_{RL}::tetR, MCS1, P_{LtetO1}::pta(LAA) (H) |
| pFT24rpt3 | P_{RL}::tetR, rhtC, pycmt, P_{LtetO1}::pta(LAA) (M) |
| pFT24rpa1 | P_{RL}::tetR, rhtC, pycmt, P_{LtetO1}::alaA(LAA) (M) |
| pFT24rpa2 | P_{RL}::tetR, rhtC, pycmt, P_{LtetO1}::alaA(LAA) (L) |
| pFT24rpa3 | P_{RL}::tetR, rhtC, pycmt, P_{LtetO1}::alaC(LAA) (M) |
| pFT24rpa4 | P_{RL}::tetR, rhtC, pycmt, P_{LtetO1}::alaC(LAA) (L) |

Notes:
Δ represents knockout; (M), (L), and (H) represent that when amplifying a target gene, the primers used are different, for example, PRL::tetR, MCS1, PLtetO1::pta (M) represents that when amplifying pta, the primer pair used is pta-Mrbs-F and pta-R; and PRL::tetR, MCS1, PLtetO1::pta(LAA) (H) represents that when amplifying pta, the primer pair used is pta-Hrbs-F and pta-LAA-R, see Table 3 for details.

TABLE 2
Sources or sequences of genes or proteins

| Gene name or | |
|---|---|
| gene fragment | Source (document, NCBI No. or sequence) |
| cI^{ts}-pR-pL | pPL451 (GenBank: AB248919.1) (Love, C. A., Lilley, P. E., Dixon, N. |
| | E., 1996.), SEQ ID NO: 12 |
| tetR | SEQ ID NO: 1, pZS4Int-1 (Lutz, R., Bujard, H., 1997. Independent |
| | and tight regulation of transcriptional units in Escherichia coli via the |
| | LacR/O, the TetR/O and AraC/I1-12 regulatory elements.) |
| P_{LtetO-1} | pZA31-luc (Lutz, R., Bujard, H., 1997. Independent and tight |
| | regulation of transcriptional units in Escherichia coli via the LacR/O, |
| | the TetR/O and AraC/I1-12 regulatory elements.), SEQ ID NO: 13 |
| MCS1 | SEQ ID NO: 2: gagctctctagaactagtggatcctgcagga |
| Terminator T7 | SEQ ID NO: 3: ctagcataaccccttggggcctctaaacgggtcttgaggggttttttg |
| MCS2 | SEQ ID NO: 4: ggaattcgatatcactcgaggtacc |
| Terminator T1 | SEQ ID NO: 5: |
| | caaataaaacgaaaggctcagtcgaaagactgggcctttcgttttatctgttgtttgtcggtgaacgctc |
| | tcctgagtaggacaaat |
| lacZ | Gene ID: 945006, SEQ ID NO: 14 |
| lacZI | Gene ID: 945006 and Gene ID: 945007, SEQ ID NO: 14 and SEQ ID |
| | NO: 15 |
| rhtC | Gene ID: 948317, SEQ ID NO: 16 |
| pyc | Sequence ID: CP002094.1 sites 625555-628968, SEQ ID NO: 17 |
| pycmt | SEQ ID NO: 6 |
| pntAB | Gene ID: 946628 and Gene ID: 946144, SEQ ID NO: 18 and SEQ ID |
| | NO: 19 |
| ppc | Gene ID: 948457, SEQ ID NO: 20 |
| aspC | Gene ID: 945553, SEQ ID NO: 21 |
| lysC | Gene ID: 948531, SEQ ID NO: 22 |
| asd | Gene ID: 947939, SEQ ID NO: 23 |
| thrA | Gene ID: 945803, SEQ ID NO: 24 |
| thrB | Gene ID: 947498, SEQ ID NO: 25 |
| thrC | Gene ID: 945198, SEQ ID NO: 26 |
| rhtA | Gene ID: 947045, SEQ ID NO: 27 |
| poxB | Gene ID: 946132, SEQ ID NO: 28 |
| pflB | Gene ID: 945514, SEQ ID NO: 29 |
| ldhA | Gene ID: 946315, SEQ ID NO: 30 |
| adhE | Gene ID: 945837, SEQ ID NO: 31 |
| pta | Gene ID: 946778, SEQ ID NO: 32 |
| tdcC | Gene ID: 947629, SEQ ID NO: 33 |
| avtA | Gene ID: 948087, SEQ ID NO: 34 |
| alaA | Gene ID: 946772, SEQ ID NO: 35 |
| alaC | Gene ID: 946850, SEQ ID NO: 36 |
| High strength | SEQ ID NO: 7: ttaaagaggagaaaggtacc |
| RBS | |
| Low strength | SEQ ID NO:8: aaacgaagcattgggatctt |
| RBS | |
| p15A | SEQ ID NO: 56 |

The disclosure is described in detail through the following examples.

### EXAMPLE 1

### Construction and Representation of Thermal Switch Vector

(1) Construction of Thermal Switch Vector

Based on a plasmid pFW001 (containing a pMB1 replicon with triclosan resistance and a high copy number), the pMB1 was replaced with a replicon p15A with a medium copy number (the nucleotide sequence was shown as SEQ ID NO: 56), and a PJ23101 promoter was replaced with a thermal switch circuit, so as to obtain the thermal switch vector. The plasmid pFW001 was derived from a paper published in 2018: Zhao, H., Fang, Y., Wang, X., Zhao, L., Wang, J., Li, Y., 2018. Increasing L-threonine production in Escherichia coli by engineering the glyoxylate shunt and the L-threonine biosynthesis pathway.

The thermal switch circuit included a temperature-sensitive circuit cIts-pR-pL, a TetR repressor gene tetR and a rigorous regulating promoter PLtetO-1. These genes were obtained through chemical synthesis. In order to realize independent epigenetic regulation, dual multiple cloning sites (MCS1 and MCS2) and different transcription terminators (T7 from phage T7 RNA polymerase and T1 from E. coli rrnB gene) were respectively inserted into two independent modules of the temperature-sensitive circuit cIts-pR-pL and a rigorous circuit tetR-PLtetO-1. The obtained thermal switch circuit was configured to successively connect a temperature-sensitive repressor gene cIts, a promoter pR-pL, the repressor gene tetR, the multiple cloning site sequence MCS1, the terminator T7, the promoter PLtetO-1, the multiple cloning site sequence MCS2 and the terminator T1 in series.

A ClonExpress MultiS one-step cloning kit (Vazyme, Jiangsu, China) was used to integrate the thermal switch circuit and the p15A replicon derived from pSU2718 onto the skeleton plasmid pFW001, so as to obtain an annular thermal switch vector. In order to make the thermal switch vector be more suitable for the gene expression control independent of the metabolic pathways in the engineering strain, the original thermal switch vector was further optimized and modified, thus generating a series of thermal switch vectors. These vectors were detected to determine whether these vectors were capable of not expressing LacZ protein under low temperature conditions (30° C.) and effectively started a gene lacZ under high temperature conditions (42° C.). Through preliminary detection and screening, it was determined that pFT22 and pFT24 had better performance.

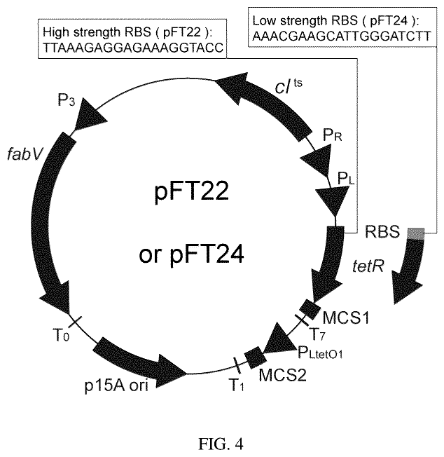

When the nucleotide sequence of the RBS (high strength) in the thermal switch circuit (the temperature-sensitive repressor gene cIts, the promoter pR-pL, the RBS, the repressor gene tetR, the multiple cloning site sequence MCS1, the terminator T7, the promoter PLtetO-1, the multiple cloning site sequence MCS2 and the terminator T1 were successively connected in series) was TTAAAGAGGAGAAAGGTACC, the thermal switch vector obtained was pFT22. When the nucleotide sequence of the RBS (low strength) in the thermal switch circuit was AAACGAAGCATTGGGATCTT (SEQ ID NO: 8), the thermal switch vector obtained was pFT24 (see FIG. 4).

(2) Representation of Thermal Switch Vector

Two reporter genes lacZ and gfp were respectively inserted onto the MCS1 and the MCS2 of the thermal switch vector (the pR-pL promoter controlled the MCS1 and the PLtetO-1 promoter controlled the MCS2) to evaluate the thermal switch system.

A primer with high strength RBS was used to amplify a lacZ fragment on the E. coli genome, and a ClonExpress II one-step cloning kit (Vazyme, Jiangsu, China) was used to insert the fragment into a SpeI enzymatic digestion site at the MCS1 of the pFT22 and the pFT24 respectively to obtain vectors pFT22-lacZ and pFT24-lacZ. Similarly, a green fluorescent protein gene gfp was integrated into an EcoRI enzymatic digestion site at the MCS2 of the vectors pFT22-lacZ and pFT24-lacZ to obtain pFT22-lacZ-gfp and pFT24-lacZ-gfp. The disclosure further constructed another recombinant expression vector, where GFP was labeled with a standard SsrA degraded peptide chain (AADENYALAA, “LAA”) to generate pFT22-lacZ-gfp (LAA) and pFT24-lacZ-gfp (LAA). The pFT22-lacZ-gfp, pFT24-lacZ-gfp, pFT22-lacZ-gfp (LAA) and pFT24-lacZ-gfp (LAA) were transformed into a lacZI cluster knockout strain TWF101 of the E. coli TWF001 to remove the background expression of lacZ on the genome.

In order to represent the capability of the thermal switch vector to regulate gene expression, a SpectraMax M3 microplate reader (Molecular Devices, USA) and a UV-1800 spectrophotometer (Shimadzu, Japan) were used to quantify the protein levels of two reporter genes on the pFT22 and the pFT24. Bacterial lawn (a transformant generated in the foregoing conversation process) on an LB agar plate was inoculated into a fresh STF seed medium, and cultured overnight at 30° C. (8 h to 14 h). The seed culture was inoculated into a 24-well culture plate with an inoculation size of initial OD600=0.4; each well contained 2 mL of fermentation medium; the seed cultures in different culture plates were placed under different culture temperature conditions (from 30° C. to 42° C.) and were cultured at 200 rpm for 4 h. To determine the sensitivity of the thermal switch system pFT24, after the switch was turned on, the change rate of the expression level of the two reporter proteins was measured.

The overnight culture at 30° C. was diluted into a 500 mL shake flask with a baffle containing 30 mL of fermentation medium at a proportion of 1:30 (V/V), and was incubated at 200 rpm under 37° C. until the OD600 reached 2.0. Then the growth condition was changed to take higher temperature (42° C.) as the starting time, and 1 mL of cell culture was collected every 20 min. The mediums mentioned above were each added with 0.9 mL of triclosan to maintain the plasmid to be stable. The collected cultures were used for fluorescent quantification in a 96-well plate by using an excitation wavelength of 488+/−10 nm and an emission wavelength of 525+/−10 nm. The cell density was detected by using the absorbancy at 600 nm, and a measured fluorescence value was standardized with OD600. The activity of β-galactosidase (LacZ) was determined with a 5 mm quartz cuvette by a colorimetric method.

In addition, the relative transcription level of the two reporter genes in the strain was detected by real-time quantitative PCR. 0.2 mL of a cell culture sample was mixed with 0.4 mL of RNAstore (Tiangen, Beijing, China), and the mixture was temporarily stored at 4° C. until processing. The total RNA of the pre-processed sample was extracted using an RNA extraction kit (BioFlux, Beijing, China), and the RNA concentration was determined using Nanodrop 2000 (Thermo Fisher Scientific, Wilmington, MA, USA). Then, HiScript II Q RT SuperMix for qPCR (+gDNA wiper) (Vazyme, Jiangsu, China) was used to remove residual DNA in the total RNA and reversely transcribe and synthesize cDNA, so as to ensure that each reaction had equal RNA for standardization. Real-time quantitative PCR was carried out on ABI Step One real-time PCR instrument (Applied Biosystems, San Mateo, CA, USA) by using ChamQ Universal SYBR qPCR Master Mix (Vazyme, Jiangsu, China), and E. coli 16S rRNA was used as an internal reference. mRNA quantification was carried out according to the specification provided by the kit. The amplification steps were as follows: predegeneration was performed at 95° C. for 30 s, then at 95° C. for 10 s, and at 60° C. for 30 s, for 40 cycles; furthermore, melting curve analysis was performed at 95° C. for 15 s, at 60° C. for 60 s, and at 95° C. for 15 s. A threshold cycle value was automatically set, and the CT value of each sample was monitored to calculate the fold difference of the relative transcription level between the two reporter genes and TWF101/pFT22 or TWF101/pFT24.

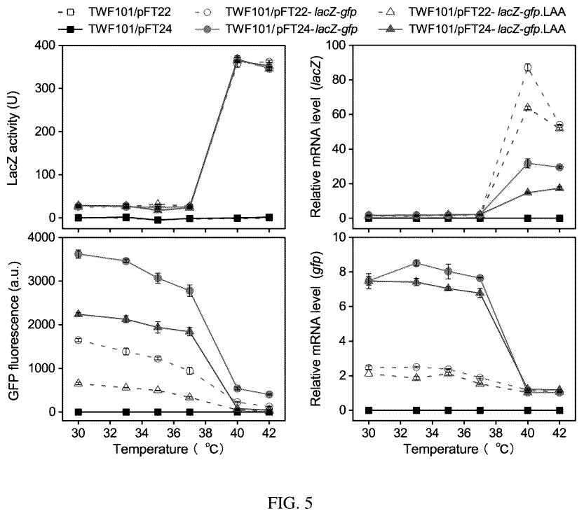

Data showed that in the pFT22 and pFT24 thermal switch systems, the expression level of LacZ was significantly increased between 37° C. and 40° C., and the enzyme activity of LacZ under higher temperature was maintained at around 350 Miller Units. Under the temperature of 37° C. or below, the pR-pL promoter was effectively inhibited by thermosensitive CI protein (as shown in FIG. 5). Therefore, the biomass accumulation of the engineering strain in the fermentation process could be set at 37° C. (the temperature suitable for E. coli growth). In the two systems, the PLtetO-1 promoter drove the expression of GFP, and would trigger the expression level to reduce when the temperature was raised. Compared with other control groups, the pFT24-lacZ-gfp (LAA) had a relatively high fluorescence value (above 2000 a.u.) at 30° C. to 37° C., and almost had no GFP protein residues at 40° C. or above.

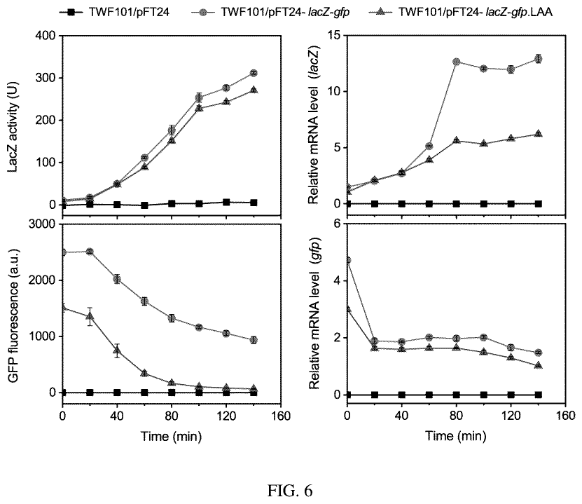

Furthermore, to detect the switch sensitivity of the pFT24 system, a strain at a logarithmic phase cultured at 37° C. was shifted to 42° C. to quantize the change of the two reporter proteins over time. In the pFT24-lacZ-gfp (LAA), the GFP fluorescence almost completely disappeared within 80 min, and at the same time, the activity of LacZ reached a fairly high level (as shown in FIG. 6). When the transcription of the gene gfp was stopped, residual GFP proteins with degradation label tails were removed more rapidly. To represent the strength of corresponding promoters in different strains, the transcription level of the reporter gene was monitored simultaneously. Data showed that the relative tendency of the mRNA level in the cell was consistent with the above protein level.

### EXAMPLE 2

### Application of Thermal Switch System in Improving Productivity of Threonine Strains

(1) Optimization and Modification of Threonine-Producing Strain

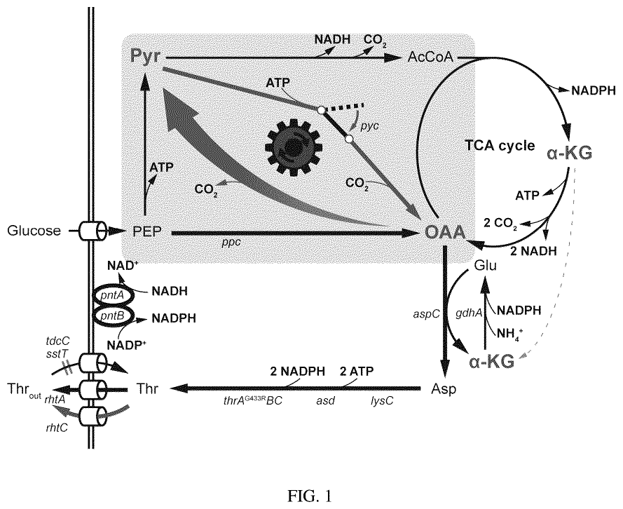

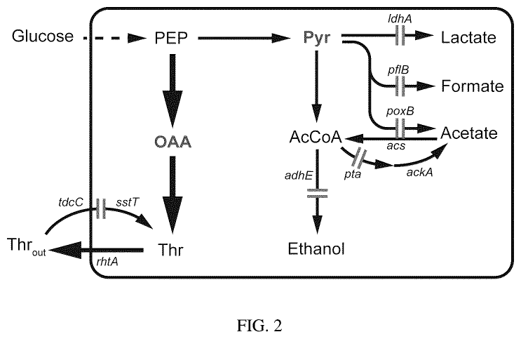

TWF001 was taken as a starting strain, and pyridine nucleotide transhydrogenase (pntAB) and related genes (ppc, aspC, lysC, asd, thrAG433RBC and rhtA) involved in threonine production had been overexpressed on the genome of the strain (see FIG. 1). In order to improve the threonine production performance of the strain and reduce carbon source waste, some unimportant genes (poxB, pflB, ldhA, adhE and pta) related to organic acid synthesis and a gene (tdcC) encoding threonine transport protein were knocked out respectively (see FIG. 2), so as to produce platform strains TWF102, TWF103, TWF104, TWF105, TWF106, TWF107 and TWF108, as shown in Table 1. These platform strains were respectively subjected to shake-flask fermentation under constant temperature of 37° C. and variable temperature (from 37° C. to 42° C.) conditions to produce threonine, so as to evaluate influence of temperature change on the capacity of the strains to produce threonine.

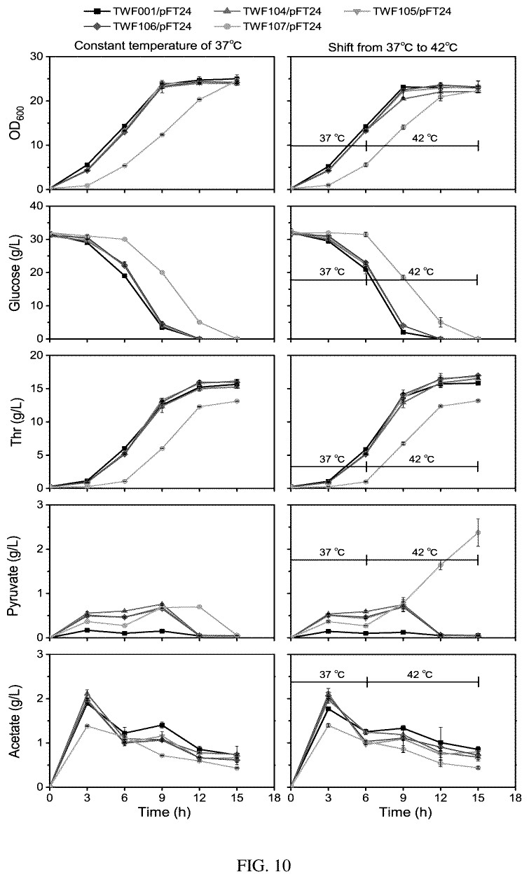

The results were shown in FIG. 10. The shake-flask fermentation data showed the tendencies of cell growth, glucose consumption, threonine yield and organic acid yield within 15 h of fermentation of different platform strains under constant temperature fermentation at 37° C. and temperature-variable (from 37° C. to 42° C.) fermentation. Firstly, compared with the condition of constant temperature fermentation at 37° C., the temperature-variable fermentation condition had no obvious negative influence on the performance of the threonine-producing strain to produce threonine. Then, by comparing the threonine yield of each strain under the temperature-variable fermentation condition, the strain TWF106/pFT24 had certain advantages. However, pta of the strain TWF107/pFT24 was knocked out, so that the growth of the strain at the early stage of fermentation was obviously inhibited, and the threonine productivity thereof was obviously reduced.

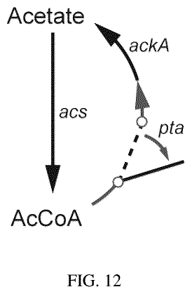

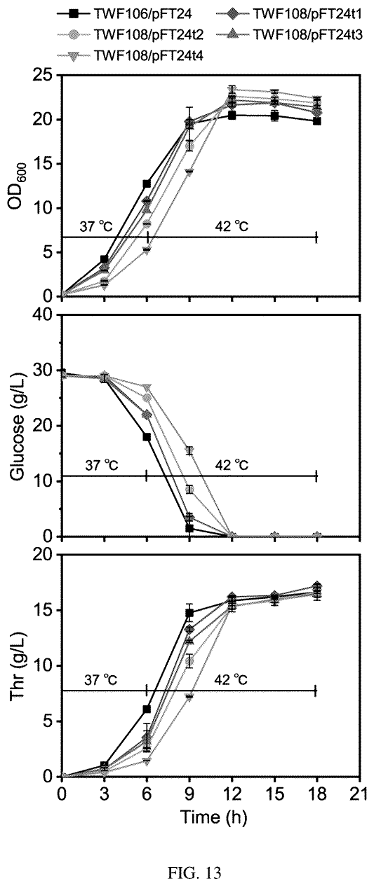

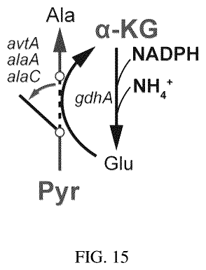

The thermal switch system was used to close some carbon source competing pathways that influenced the strain growth, including acetic acid synthetic pathways and L-alaine synthetic pathways. In order to construct an acetic acid synthetic pathway closing system, the pta gene encoding phosphate acetyltransferase on the genome of the threonine-producing strain was knocked out. After the pta gene was inserted into the PLtetO-1 promoter, expression vectors pFT24t1, pFT24t2, pFT24t3, pFT24t4 and pFT24rpt3 were obtained; and the PLtetO-1 promoter controlled the pta gene to be normally expressed at the cell growth stage and stop the expression at the fermentation production stage, so as to save carbon sources (as shown in FIG. 12). As shown in FIG. 13, the expression vectors pFT24t1, pFT24t2, pFT24t3, pFT24t4 and pFT24rpt3 were transferred into the strain TWF108 with pta being knocked out, but compared with TWF106/pFT24, the threonine productivity was not improved. Similarly, the L-alaine synthetic pathway transformed pyruvate into L-alaine through L-alaine synthetic transaminase, including three main proteins: AvtA, AlaA and AlaC. Therefore, a multi-knockout strain TWF113 (TWF106ΔavtAΔalaAΔalaC) was constructed, and was used for further designing a thermal switch system for closing L-alaine synthesis. Genes alaA and alaC containing different RBS sequences (see Table 3) were respectively inserted into the PLtetO-1 promoter to obtain pFT24rpa1, pFT24rpa2, pFT24rpa3 and pFT24rpa4, and the thermal switch vector pFT24 controlled the genes to perform gene expression at the strain growth stage to recover normal growth and biomass accumulation of the strain (as shown in FIG. 15). At the fermentation stage, these pathways were closed to reduce shunting of excessive carbon sources to other pathways, so as to concentrate more carbon sources to synthesize the target product.

(2) Optimization and Modification of Thermal Switch Vector

In order to verify the dynamic regulation effect of the thermal switch system on the central metabolic pathway, a heterologous gene pyc was amplified from Lactococcus lactis using a primer with a high strength RBS sequence. Similarly, a gene rhtC with a high strength RBS sequence was obtained by amplification from an E. coli TWF001 genome and the primer used was shown in Table 3. A codon-optimized synthetic gene pycmt as well as genes pyc and rhtC were respectively inserted into a SpeI site of the thermal switch vector pFT24 by using a one-step cloning kit, and the site was located at the downstream of the pR-pL promoter. The two genes rhtC and pycmt were co-expressed under the control of the cIts-pR-pL circuit to obtain a thermal switch vector pFT24rp.

These thermal switch vectors were respectively introduced into an engineering strain TWF106 through electrotransformation to produce a series of recombinant strains. In order to improve the threonine production efficiency in engineering microbiology, it was attempted to migrate several key genes of a competing pathway that influenced the cell growth from the E. coli chromosome to the thermal switch vector. Pta, gltA, alaA and alaC were respectively amplified from the E. coli genome with a primer appended with an “LAA” degraded tail, and were respectively cloned to an EcoRI restriction site of the vector pFT24 or pFT24rp. The enzymatic digestion site was at the downstream of the PLtetO-1 promoter. Complementary plasmids containing the target genes with different strengths of RBS were introduced into the target strain to recover the cell growth.

TABLE 3
Primers

| Primer | Sequence (capital letters are the RBS sequence) | |
|---|---|---|
| rhtC-rbs-F | tggagctctctagaactagATAAGAGGTATATATTAatgttg | SEQ ID |
| | atgttatttctcaccgt | NO:37 |
| rhtC-R | cttcctgcaggatccactagtcgccttatccgacttactctg | SEQ ID |
| | | NO:38 |
| pyc-rbs-F | tggagctctctagaactagAAGGAGATATACATatgaaaaaac | SEQ ID |
| | tactcgtcgccaat | NO:39 |
| pycmt-rbs-F | tggagctctctagaactagAAGGAGATATACATatgaaaaaac | SEQ ID |
| | tgctggtcgcaaat | NO:40 |
| pycmt-Crbs-F | aagtcggataaggcgactagAAGGAGATATACATatgaaaaa | SEQ ID |
| | actgctggtcgcaaat | NO:41 |
| pyc-R | cttcctgcaggatccactagttacgtcaaagttagtcaatttcaatcaataagtc | SEQ ID |
| | | NO:42 |
| pta-Mrbs-F | tgaccgaatacattggaattgtgctgttttgtaacccgcc | SEQ ID |
| | | NO:43 |
| pta-Hrbs-F | tgaccgaatacattggaatTACACAGGAAACCTACTAGgt | SEQ ID |
| | gtcccgtattattatgctgatcc | NO:44 |
| pta-R | acctcgagtgatatcgaattcactgcggatgatgacgagat | SEQ ID |
| | | NO:45 |
| pta-LAA-R | acctcgagtgatatcgaattcattaagctgctaaagcgtagttttcgtcgtttgc | SEQ ID |
| | tgcctgctgctgtgcagactgaatcgcagt | NO:46 |
| gltA-Hrbs-F | tgaccgaatacattggaatTAAAGAGGAGAAAGGATTCat | SEQ ID |
| | ggctgatacaaaagcaaaactcacc | NO:47 |
| gltA-Mrbs-F | tgaccgaatacattggaattacgcaataaggcgctaagg | SEQ ID |
| | | NO:48 |
| gltA-LAA-R | acctcgagtgatatcgaattcattaagctgctaaagcgtagttttcgtcgtttgc | SEQ ID |
| | tgcacgcttgatatcgcttttaaagtcgc | NO:49 |
| alaA-Mrbs-F | tgaccgaatacattggaattggagtacattgttctaagctgacttc | SEQ ID |
| | | NO:50 |
| alaA-Lrbs-F | tgaccgaatacattggaatTACACAGGAAACCTACTAGat | SEQ ID |
| | gtcccccattgaaaaatccag | NO:51 |
| alaA-LAA-R | acctcgagtgatatcgaattcattaagctgctaaagcgtagttttcgtcgtttgc | SEQ ID |
| | tgccagctgatgataaccagaaagg | NO:52 |
| alaC-Mrbs-F | tgaccgaatacattggaattggacagacagaaattaatcaggctat | SEQ ID |
| | | NO:53 |
| alaC-Lrbs-F | tgaccgaatacattggaatTACACAGGAAACCTACTAGat | SEQ ID |
| | ggctgacactcgccctgaacg | NO:54 |
| alaC-LAA-R | acctcgagtgatatcgaattcattaagctgctaaagcgtagttttcgtcgtttgc | SEQ ID |
| | tgcttccgcgttttcgtgaatatgt | NO:55 |

### EXAMPLE 3

### Fermentation Production of Threonine

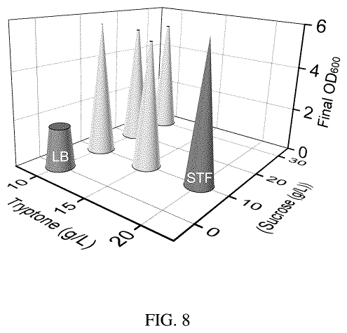

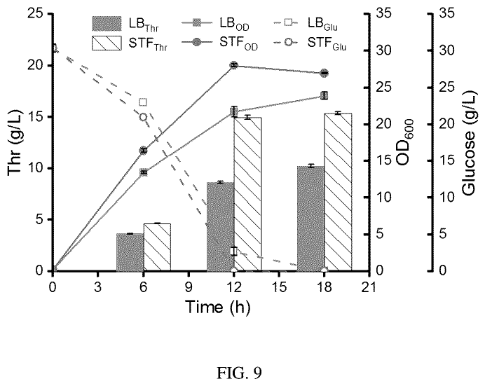

An STF seed medium was obtained through optimization and improvement by taking an LB medium as the basis. The saccharose and peptone contents in the LB medium were regulated to culture the TWF001 strain, and the growth condition and threonine yield of the strain in the seed medium with different components were detected (as shown in FIG. 8 and FIG. 9). The result showed that by taking the STF seed medium as the seed medium and performing shake-flask fermentation at constant temperature of 37° C., after the fermentation was performed for 18 h, the threonine yield of the TWF001 was 15.37 g/L, which was increased by 50.54% compared with the situation that the LB was used as the seed medium.

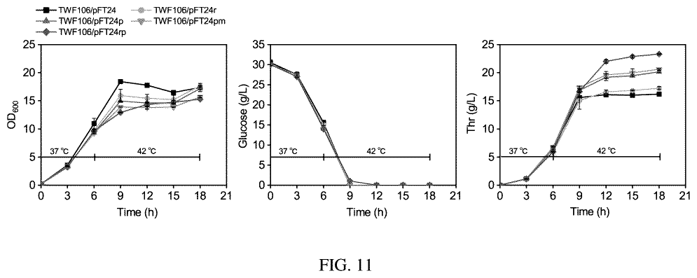

After temperature-variable shake-flask fermentation (as shown in FIG. 11), the threonine yield of the recombinant strains TWF106/pFT24r, TWF106/pFT24p, TWF106/pFT24pm and TWF106/pFT24rp were respectively 17.24 g/L, 20.15 g/L, 20.60 g/L and 23.29 g/L, and the corresponding molar glucose-acid conversion rates were respectively 72.44%, 81.50%, 98.43% and 111.78%.

In the fermentation process of threonine, the accumulation of acetic acid might influence the normal growth of the strain and reduce the threonine productivity of the strain at the same time. Cutting off the acetic acid production pathway by conventional knockout of pta would seriously inhibit the growth of the strain, thereby reducing the capability of the strain to produce threonine (as shown in FIG. 10). We tried to stop the expression of pta through the thermal switch system, so as to save the carbon sources and reduce the formation of acetic acid. The threonine shake-flask fermentation data showed that compared with TWF106/pFT24rp, although the final strain biomass was increased by stopping the pta expression, the threonine productivity of the strain was not further improved.

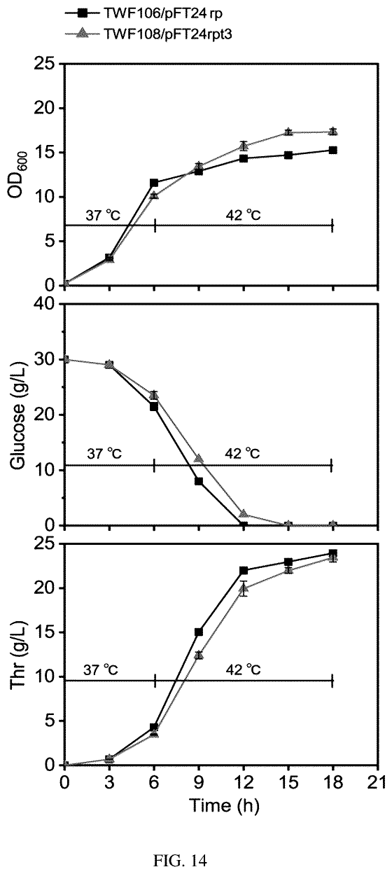

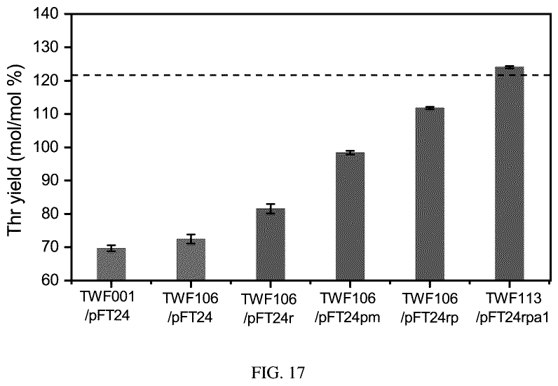

On the basis of the strain TWF106/pFT24rp with high threonine yield, the alanine synthetic pathway was further closed by using the thermal switch vector to obtain a recombinant strain TWF113/pFT24rpa 1 (as shown in FIG. 14). The specific steps were as follows: on the basis of the TWF106, three genes avtA, alaA and alaC were simultaneously knocked out to obtain a strain TWF113; on the basis of a thermal switch vector pFT24rp, the expression of alaA was controlled by the promoter PLtetO-1 to obtain an expression vector pFT24rpa1. After temperature-variable shake-flask fermentation, the threonine yield of the recombinant strain TWF113/pFT24rpa1 reached 25.85 g/L, and the molar glucose-acid conversion rate was 124.03% (as shown in FIG. 17). The organic acid formation conditions of all the strains in the process of shake-flask fermentation were detected, and it was found that pyruvate and acetic acid generated in the early stage of fermentation of the strains could be reabsorbed and utilized after the thermal switch system was turned on.

Although the disclosure has been disclosed with the above preferred examples, these examples are not intended to limit the disclosure. Anyone skilled in the art can make various changes and modifications without departing from the spirit and scope of the disclosure. Therefore, the protection scope of the disclosure should be defined by the claims.

What is claimed is:

1. A thermal switch vector, obtained by connecting a thermal switch circuit onto a vector, wherein the thermal switch circuit comprises a temperature-sensitive circuit cI^{ts}-p_{R}-p_{L} and a rigorous circuit tetR-P_{LtetO-}1; the temperature-sensitive circuit cI^{ts}-p_{R}-p_{L} is composed of a temperature-sensitive repressor gene cI^{ts} and a tandem promoter P_{R}-P_{L} and a nucleotide sequence is shown as GenBank: AB248919.1; the rigorous circuit tetR-P_{LtetO-}1 is composed of a repressor gene tetR and a promoter P_{LtetO-1}, a nucleotide sequence of the repressor gene tetR is shown as SEQ ID NO: 1; pMB1 in a plasmid pFW001 is replaced with a replicon p15A with a medium copy number, and a PJ23101 promoter in the plasmid pFW001 is replaced with the thermal switch circuit; the thermal switch circuit is configured to successively connect the temperature-sensitive repressor gene cI^{ts}, the promoter P_{R}-P_{L}, RBS, the repressor gene tetR, a multiple cloning site sequence MCS1, a terminator T7, the promoter P_{LtetO-1}, a multiple cloning site sequence MCS2 and a terminator T1 in series; the RBS is high strength RBS or low strength RBS, a nucleotide sequence of the high strength RBS is shown as SEQ ID NO: 7, and a nucleotide sequence of the low strength RBS is shown as SEQ ID NO: 8.

2. The thermal switch vector according to claim 1, wherein when the RBS is the low strength RBS, genes rhtC and pycmt are successively inserted at the MCS1 of the vector, and the genes rhtC and pycmt are co-expressed under the control of the cI^{ts}-p_{R}-p_{L} circuit to obtain a thermal switch vector pFT24rp; or when the RBS is the low strength RBS, a gene rhtC or a gene pyc is inserted at the MCS1 of the vector to obtain pFT24r and pFT24p respectively.

3. The thermal switch vector according to claim 2, wherein on the basis of the thermal switch vector pFT24rp, a gene alaA labeled with a standard SsrA degraded peptide chain is inserted in the MCS2 to obtain a thermal switch vector pFT24rpa1; and an amino acid sequence of the standard SsrA degraded peptide chain shown as SEQ ID NO: 10.

4. A threonine-producing strain, expressing the thermal switch vector according to claim 1.

5. The strain according to claim 4, wherein the thermal switch vector according to claim 3 is transferred to a threonine-producing platform strain, and the threonine-producing platform strain comprises Escherichia coli TWF001, TWF101, TWF102, TWF103, TWF104, TWF105, TWF106, TWF107, TWF108, TWF110, TWF111, TWF112 or TWF113.

6. The strain according to claim 5, wherein the E. coli TWF106 is obtained by knocking out poxB, pflB, ldhA, adhE and tdcC in the TWF001.

7. The strain according to claim 5, wherein the E. coli TWF113 is obtained by knocking out poxB, pflB, ldhA, adhE, tdcC, avtA, alaA and alaC in the TWF001.

8. A method for producing threonine, wherein threonine is produced by taking the strain according to claim 7 as a fermentation strain.

9. The method according to claim 8, wherein a fermentation strain seed culture with an initial OD_{6 00} of 0.2 to 0.3 is inoculated into a fermentation medium, fermentation culture is performed at 36° C. to 38° C. for 5 h to 8 h, and culture is continued at 41° C. to 43° C. until glucose in a fermentation broth is completely consumed.

10. The method according to claim 9, wherein the fermentation strain seed culture is obtained by culturing the strain in an STF seed medium; and the STF seed medium contains 10 g/L saccharose, 20 g/L peptone, 5 g/L yeast extract, 15 g/L (NH₄)₂ SO₄ and 1 g/L MgSO4, with the pH being regulated to 7.3.
